(19) United States
(12) Patent Application Publication
(10) Pub. No.: US 2009/0298952 A1
(43) Pub. Date: Dec. 3, 2009

(54) PLATABLE SOLUBLE DYES

(76) Inventors: Karen S. Brimmer, St. Louis, MO (US); Penny F. Martin, St. Louis, MO (US)

Correspondence Address: MICHAEL BEST & FRIEDRICH LLP, 100 E WISCONSIN AVENUE, Suite 3300, MILWAUKEE, WI, 53202, US

(21) Appl. No.: 12/437,219

(22) Filed: May 7, 2009

Related U.S. Application Data
(60) Provisional application No. 61/051,226, filed on May 7, 2008.

(51) Int. Cl. A23L 1/27; A61K 47/04

(52) U.S. Cl. 514/770; 426/540; 426/250; 424/49; 426/531

(57) ABSTRACT

Edible colored powders are formed by combining silica with a soluble dye, such as a natural dye. The colored powders can be used to color edible substrates such as particles and compressed tablets. Methods of forming the edible colored powders may include a drying step to reduce the moisture content of the colored powder.

### CROSS-REFERENCE TO RELATED APPLICATIONS

This application claims the benefit of priority to U.S. Patent Application No. 61/051,226, filed May 7, 2008, the disclosure of which is herein incorporated by reference in its entirety.

### INTRODUCTION

Edible coloring agents, such as pigments, dyes and lakes, are commonly used in industry and commerce to add color to food, pharmaceuticals or other edible consumer products. For many applications in which moisture or other liquid is deleterious to the edible product, it is desirable to use a coloring agent in dry form. A dry form of a soluble colorant may be produced by combining the colorant with an aluminum salt, zinc salt or earth metal salt to form a lake. However, for many consumer applications, the use of such metal salts is undesirable.

### SUMMARY OF THE INVENTION

In one aspect, the invention provides a colored powder comprising silica and a soluble dye. The powder may be substantially free of aluminum salts.

In another aspect, the invention provides a method of coloring an edible substrate comprising plating a colored powder substantially free of aluminum salts and comprising silica and a soluble dye onto the edible substrate.

In another aspect, the invention provides an edible solid substrate comprising a colored powder comprising silica and a soluble dye adsorbed onto the silica.

In another aspect, the invention provides a method of coloring an edible substrate, comprising combining a colored powder comprising silica and a soluble dye adsorbed onto the silica with an edible substrate.

In another aspect, the invention provides a method of increasing the color intensity of a dye. A solution of a soluble dye is adsorbed onto silica powder to form a colored powder and the colored powder is dried using heat, a vacuum, air or gas flow or a combination thereof.

### DETAILED DESCRIPTION

Before any embodiments of the invention are explained in detail, it is to be understood that the invention is not limited in its application to the details of construction and the arrangement of components set forth in the following description. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The present invention relates to dyes, and more particularly to forms of natural or synthetic dyes that have plating characteristics for use in foods or pharmaceuticals. In one aspect, the invention provides a colored powder containing silica and a soluble dye, such as a soluble natural dye, that has the capacity to coat or be disposed upon edible substrates such as edible particles. In another aspect, the invention provides an edible colored powder comprising silica and a soluble dye adsorbed onto the silica, wherein the colored powder is substantially free of one or more alkaline earth metal (e.g. magnesium, calcium, barium) salts, iron salts, aluminum salts and/or zinc salts. In another aspect, the invention provides an edible substrate, such as edible particles, which includes a colored powder comprising silica and a soluble dye adsorbed onto the silica. In one aspect, the edible substrate is a compressed tablet, and/or contains a pharmaceutical. In one aspect, the invention provides a method of increasing the color intensity of a dye, such as a natural or synthetic dye, in dry form by adsorbing a solution of the dye onto silica powder. In another aspect, the invention provides a method of coloring an edible substrate by combining a colored powder comprising silica and a soluble dye adsorbed onto the silica with an edible substrate.

The inventors surprisingly discovered that when a solution of a natural or synthetic dye is combined with silica, a more intense color is obtained. Accordingly, less dye is required to produce a particular color intensity when the dye is combined with silica, than when the dye is not combined with silica. For example, less than about 60%, less than about 70%, less than about 80%, less than less than about 85%, less than about 90%, or less than about 95% of an amount of dye may be required, when combined with silica, to produce a color intensity comparable to the color intensity of the dye used without silica. For example, a beta carotene and silica blend containing about 0.14% beta carotene used at 0.8% by weight in a sucrose mixture may produce a yellow shade that is equivalent to a similar amount of turmeric powder containing about 1.6% curcumin. Less dye may be used with an edible substrate to provide a desired color.

The use of silica also facilitates the deposition of the dye in a dry form, such that natural or synthetic dyes may be used in a manner comparable to lake pigments. For example, beta carotene, beet and anthocyanins adsorbed in a soluble form to the silica may be plated using the techniques described herein.

The dye is suitably adsorbed by the silica, such that the liquid or solution of dye coats the external and internal surfaces of the silica particle. When the silica is dried, a colored powder is obtained which can be disposed onto an edible substrate. The colored powder may also suitably be used to color compressed edible tablets or to color liquids in which the dye is soluble. The dye remains in a soluble form in the colored powder, and will be released from the silica into solution when mixed or added to a solvent. The solvent may include, for example, water, one or more alcohols (such as ethanol, propanol, butanol, etc.), glycerin, glycols (such as propylene glycol), or a combination thereof.

The colored powder is suitably food grade. As used herein, “food grade” means that up to specified amounts of the colored powder or individual ingredients making up the colored powder can be ingested by a human without generally causing deleterious health effects. Therefore, in order to meet the standard of a “food grade” colored powder, the colored powder should be free or substantially free of ingredients that generally cause deleterious health effects when ingested by a human. When such ingredients are present, e.g., in trace amounts through contamination, those ingredients should be present in amounts below those that would result in the deleterious health effects. Examples of food grade ingredients include those ingredients “generally recognized as safe” (“GRAS”) by the United States Food and Drug Administration (“FDA”) and flavors and colors approved by the FDA for use in foods for human consumption. In particular, food safe ingredients include those ingredients listed as approved under 21 C.F.R. §§172.510, 172.515, 172.520, 172.530, 172.535, 172.575, 172.580 and 172.585. Suitably the dye is Kosher.

Natural dyes that may be used in colored powders of the invention include those disclosed in 21 C.F.R. § 73 as being suitable for food or drug use. Suitable natural dyes include those listed as being exempt from certification by the FDA. Natural dyes may be derived or extracted from natural sources, or may be synthetic duplicates of such substances. Examples of suitable natural dyes include, without limitation, caramel color, turmeric, turmeric oleoresin, annatto, beta carotene, paprika, paprika oleoresin, red cabbage extract, beet extract, grape skin extract, gardenia extracts, cochineal extracts including carminic acid or carmine, saffron, tomato lycopene extract, riboflavin, chlorophyll-containing extracts, such as nettle extract, alfalfa extract and spinach extract, and other natural colors derived from vegetable juices. Anthocyanins are another class of food grade natural dyes that may be used in the colored powders. The anthocyanins may be derived from a variety of plant sources, including fruit juices, elderberries, blackcurrants, chokeberries, vegetable juices, black carrots, red cabbage, grapes and grape skins, and sweet potatoes, such as purple sweet potatoes.

Suitably, synthetic colors or dyes may also be combined with silica to form a colored powder. Synthetic dyes may be added to the silica in a liquid form to provide a highly colored, platable powder. Synthetic dyes include, without limitation, azo dyes (for example, Yellow 5, Yellow 6, Red 40, Carmoisine, Amaranth, Ponceau 4R), Blue 1, Blue 2, Patent Blue V, Red 3, Green 3, Brilliant Black BN, and Quinoline Yellow. For example, a Red 3 bright pink shade may be produced by adsorbing Red 3 onto silica to produce a colored powder, and applying the Red 3/silica colored powder onto or into a food or pharmaceutical.

Although the relative amount of the food grade dyes used in the colored powders may vary depending on the desired color, shade and intensity, the food grade colored powders, when used, will typically comprise at least about 0.1 wt. % or at least about 0.2 wt. % (dry solids basis, hereinafter dsb) food grade dye, in others at least about 0.3 wt. % or at least about 0.5 wt. % (dsb) food grade dye, and in yet others at least about 0.75 wt. % or at least about 1 wt. % (dsb) food grade dye. In some embodiments, the colored powders comprise less than about 10 wt. % or less than about 5 wt. % (dsb) food grade dye, in others less than about 4 wt. % food grade dye, and in yet others less than about 3 wt. % (dsb) food grade dye. This includes embodiments where the colored powders contain about 0.1 to about 5 wt. % (dsb), and further includes embodiments where the food grade colored powders contain about 0.2 to about 3 wt. % (dsb) food grade dye.

The colored powder may contain one or more natural or synthetic dyes. Suitably, the colored powder comprises at least about 1 (or at least 1), at least about 2 (or at least 2) or at least about 3 (or at least 3) natural or synthetic dyes, or a combination thereof.

In one embodiment, the food grade colored powder excludes synthetic dyes, or contains (dsb) less than about 0.1 wt. %, less than about 0.01 wt. %, less than about 0.001 wt. %, less than about 0.0001 wt. %, or less than about 0.00001% synthetic dye. Suitably, the food grade colored powder is substantially free of one or more alkaline earth metal (e.g. magnesium, calcium, barium) salts, iron salts, aluminum salts and/or zinc salts. Illustrative salts include chloride, acetate, nitrate, oxide, hydroxide, carbonate, sulfate, or phosphate salts, as well as oxides or hydroxides. For example, the food grade colored powder may be substantially free of one or more of aluminum sulfate, aluminum hydroxide, aluminum oxide, barium sulfate or calcium sulfate. The food grade colored powder may contain (dsb) less than about 0.1 wt. %, less than about 0.01 wt. %, less than about 0.001 wt. %, less than about 0.0001 wt. % or less than about 0.00001 wt. % of one or more of the metal salts set forth above.

Silica that may be suitably used in the colored powders includes, without limitation, precipitated silica, gel silica and fumed silica. Gel and precipitated silica are formed by “wet chemistry” processes and form a three-dimensional network of particles or aggregates. The increased surface area provided by this three-dimensional network permits gel silica and precipitated silica to adsorb and immobilize the natural or synthetic dye. Suitable precipitated silicas include the SIPERNAT® series (commercially available from Evonik Degussa, GmbH, Germany), such as SIPERNAT® 22, 22 LS, 22S, 33, 50, 50S, and 500 LS, and ZEOTHIX® silicas (commercially available from J.M. Huber Corporation, New Jersey, U.S.), such as ZEOTHIX® 265 and ZEOTHIX® 95. The shade, hue and strength of color in the colored powders may vary depending on the size of the silica particles used.

Fumed silica particles can be produced by pyrogenic processes and have the chemical composition SiO₂. Fumed silica particles, typically, are aggregate particles of smaller primary particles, which are held together by relatively strong cohesive forces, such that the aggregate particles are not broken down into primary particles when dispersed in a liquid medium. Aggregate fumed silica particles may also form larger agglomerate particles, which are held together by relatively weak cohesive forces.

Suitable silica particles have a size of at least about 1 μm, at least about 2 μm, at least about 4 μm, at least about 5 μm, at least about 7 μm and less than about 300 μm, less than about 300 μm, less than about 250 μm, less than about 200 μm, less than about 175 μm, less than about 150 μm. Suitably the silica particles may show a high oil adsorption. For example, the silica particles may have a DBP (dibutyl phthalate) oil adsorption of at least about 150, at least about 175, at least about 200, at least about 225, or at least about 250 g per 100 gram silica.

In some embodiments, the colored powders may contain one or more additives such as ascorbic acid; antioxidants; stabilizers; tocopherols; sodium hexametaphosphate; ascorbyl palmitate; food grade edible oils such as soybean, cottonseed, palm and sunflower oils; preservatives; emulsifying agents such as Polysorbate 80 and lecithin; sequestrants; pH modifiers such as metal hydroxides (for example Ca(OH)₂, NaOH and KOH), citric acid; ethylene-diamine-tetra-acetic acid (EDTA); tetrasodiumpolyphosphate (TSPP); carbonate salts (for example such as calcium carbonate, potassium carbonate and sodium carbonate); sodium citrate; calcium polyphosphate; film formers and hydrocolloids such as Gum Arabic; stearic acid, food grade shellacs and film layerants such as metallic stearates; parting agents such as cellulose and fumed silica and modified starches, such as maltodextrin. These additives are typically present in small quantities, for example, no more than about 10 wt. % and commonly no more than about 5 wt. %.

The invention provides methods of making a colored powder by combining silica with a dye, such as a natural dye. Suitably, the dye is in a solution, extract, or other liquid form. The dye may be combined with the silica by, for example, spraying, pouring or adding drop-wise to the silica, such that the dye is suitably gradually combined with the silica. For example, liquid ingredients may be sprayed through nozzles onto the silica in the blender and blended until homogenous. Suitably the silica is agitated, for example by mixing, blending, or rotating as the dye is added. For example, a tumble mixer, such as a Nauta mixer, e.g. a vertical Nauta mixer, a ploughshare mixer, a commercial blender, such as a LITTLEFORD DAY® blender in which the choppers are removed, or a horizontal ribbon blender, may be used. Suitably, the liquid dye or dye solution is sprayed into the mixer or blender through atomizer nozzles. Addition of the liquid ingredients may occur, for example, via a pressure pot pressurized with compressed air through the nozzles, or from an atmospheric mixing vessel fed to the mixer spray nozzles with a pump such as a peristaltic metering pump.

Additives may also be similarly combined with the silica, either before or after adding the dye, or by adding together with the dye. Additives may be combined with silica, for example, by spraying, pouring or adding drop-wise to the silica. One or more additives may be combined with the dye, or with each other, prior to being combined with the silica. One or more additives or dyes may also be added sequentially to the silica. Suitable conditions under which the dye is added may be selected such that a homogeneous colored powder is obtained. In one embodiment, a natural dye such as beta carotene is added to the silica, and then stabilizers and other additives are added. In one embodiment, to obtain a red shade, anthocyanin (from, for example, red cabbage or purple sweet potato) is first added, followed by stabilizers or other additives. In one embodiment, to obtain a purple shade, anthocyanin is added after stabilizers, pH modifiers or other additives are added to the silica.

The silica and dye may be dried to reduce the moisture content of the colored powder. Drying may be accomplished, for example, by heating, air or gas flow, and/or by using a vacuum treatment during or after the mixing process. For example, a suitable mixer such as a ploughshare, vertical Nauta or horizontal ribbon blender may be fitted with a low pressure jacket which is supplied with steam or another suitable transfer fluid to provide heating capability for drying the product. Suitably the mixer used has lower temperature vacuum drying capability.

Liquid ingredients may be sprayed through nozzles onto the powder in the blender and blended until homogenous. In one embodiment, addition of the liquid ingredients is achieved via a pressure pot pressurized with compressed air through the nozzles or from an atmospheric mixing vessel fed to the mixer spray nozzles using a pump (for example, a peristaltic metering pump). Low pressure steam, e.g., 15 psi, may be supplied to the mixer jacket to be used as the energy source for drying.

The blend may be dried at a temperature of at least about 20° C., at least about 25° C., at least about 30° C., at least about 35° C., at least about 40° C. or at least about 45° C., and less than about 90° C., less than about 85° C. less than about 80° C. less than about 75° C. less than about 70° C., less than about 65° C., less than about 60° C., or less than about 55° C. The drying process may be carried out for a period of at least about 2 minutes, at least about 5 minutes, at least about 7 minutes, at least about 10 minutes, or at least about 15 minutes, at least about 30 minutes and less than about 5 hours, less than about 2 hours, less than about 90 minutes and less than about 1 hour. For example, the drying may be carried out for about a period of about 15 minutes to about 90 minutes.

The total moisture content of the colored powder by weight is suitably reduced in the drying step to at least about 20%, at least about 30%, at least about 40%, at least about 50%, at least about 60%, at least about 70%, at least about 80%, or at least about 90% of the total moisture content of the colored powder prior to drying. After drying or otherwise, the total moisture content of the colored powder is suitably less than about 20%, less than about 15%, less than about 10%, less than about 9%, less than about 8%, less than about 7%, less than about 6%, less than about 5%, less than about 4%, or less than about 3% and at least about 0.01%, at least about 0.05%, at least about 0.1%, at least about 1% or at least about 2% by total weight of the colored powder. For example, the moisture content may be at least about 2% and less than about 10% of the total weight of the colored powder

The colored powders of the present invention are stable and retain color when stored. As used herein, “stable” means that the colored powders show no visible sign of microbial contamination after a period of storage at a predetermined temperature. Microbial contamination may be evident as visible mold or slime, or visible as an increase in turbidity when the colored powder is mixed with water. Colored powders are suitably stable after storage at room temperature (about 21° C.) or refrigeration (about 4° C.) for period of at least at least about 1 month, at least about 2 months, at least about 4 months, at least about 6 months, at least about 9 months, at least about 12 months, at least about 2 years, at least about 3 years, at least about 4 years or at least about 5 years.

As used herein, “retains color” means that 80% of the initial color intensity is present after a period of storage at a predetermined temperature, measured using a calorimeter. The initial color intensity and the intensity after a period of storage are each measured against a standard. Colored powders suitably retain color after storage at room temperature (about 21° C.) or refrigeration (about 4° C.) for period of at least about 1 month, at least about 2 months, at least about 4 months, at least about 6 months, at least about 9 months, at least about 12 months, or at least about 2 years. In some embodiments, at least about 85%, at least about 90%, or at least about 95% of the initial color intensity is present after storage under the aforementioned conditions.

Suitably, the colored powder contains a dye solution added in amount of at least about 0.25 parts, at least about 0.5 parts, at least about 1 part, or at least about 1.5 parts per 100 parts silica. Suitably the colored powder contains dye solution added in amount of less than about 10 parts, less than about 5 parts, less than about 4 parts or less than about 3 parts per 100 parts silica.

Suitably, the colored powder contains a dye in an amount of at least about 0.01 parts, at least about 0.02 parts, at least about 0.05 parts, at least about 0.1 parts, or at least about 0.15 parts per 100 parts silica. Suitably the colored powder contains a dye in an amount of less than about 25 parts, less than about 23 parts, less than about 20 parts, less than about 19 parts, or less than about 18 parts per 100 parts silica. For example, for some dyes such as beta carotene and betanin (from, for example, beets), the colored powder contains the dye in an amount of less than less than about 5 parts, less than about 4 parts, less than about 3 parts, less than about 2.5 parts, less than about 2 parts, or less than about 1.5 parts per 100 parts silica.

The colored powders are suitable for being disposed on, mixed with or coated onto a wide variety of edible substrates. As used herein, “edible substrate” or “substrate” includes any material suitable for consumption that is capable of having a colored powder disposed, coated, or plated thereon, or that can be colored upon mixing with one or more colored powders. Suitably the edible substrate is a solid edible substrate. Examples of edible substrates onto which the food grade colored powders may be disposed include snack chips (e.g., sliced potato chips), fabricated snacks (e.g., fabricated chips such as tortilla chips, potato chips, potato crisps, extruded snacks), candy (e.g., chewing gum, chocolate, hard candy, compressed candy, candy chews, marshmallows, taffy, pellet gum), fruit (e.g., fresh fruit, dried fruit, dried fruit film, processed fruit snacks), processed meat (e.g., beef jerky, hot dogs, sliced meats, meat patties, fish sticks), cheese, desserts (e.g., pudding, ice cream, gelatin, ice cream sandwiches, ice pops, yogurt), baked products (e.g., bread products (e.g., biscuits, toast, buns, bagels, English muffins, and tortillas), ice cream cones, cookies, cakes (e.g., pancakes, cheese cake, cup cakes, iced cakes, muffins), pizza, pies, pretzels, crackers, waffles, breakfast cereals, toaster pastries, vanilla wafers) and pasta. Additional examples of edible substrates may include, but are not limited to, pharmaceutical applications, nutraceutical applications (such as antacids, vitamins, etc.), capsules, tablets, pet food applications (e.g., pet treats, snacks, and food), and oral hygiene applications (e.g., toothpastes and mouthwashes).

Additional examples of edible substrates may include, but are not limited to, edible particles, such as maltodextrin, sugar (such as glucose, sucrose, fructose, maltose, lactose), starch (such as corn starch, wheat flour, barley flour, oat flour, rye flour, tapioca flour) dextrose, sorbitol, spices, seasonings, salt (such as sodium chloride, potassium chloride), sodium starch glycolate, cellulose, calcium monohydrogen phosphate, calcium sulfate, dicalcium phosphate, calcium carbonate, light anhydrous silicic acid, titanium oxide, and magnesium aluminometasilicate. Suitably the edible particles combined with the colored powder may themselves be disposed on or used in other edible substrates. Suitably, the additional components such as binders and lubricants may be added to the edible particle and colored powder mixtures. For example, lubricants such as stearic acid (stearin), hydrogenated oil, and sodium stearyl fumarate may be used. Although any substrate may be combined with any food grade colored powder, some substrates may be more compatible than others with a particular food grade colored powder.

Suitably, the colored powder containing silica and a soluble dye has plating characteristics and may be plated onto the edible substrate. As used herein, “plating” or “plating characteristics” means that when the edible substrate is contacted with the colored powder, the color of the dye is visually evident where the colored powder contacts the edible substrate. For example, when a colored powder containing silica and beta carotene, or silica and anthocyanin, is mixed with an edible substrate such as dextrose, the dextrose takes on a yellow or red color. In contrast, when a dry form of the dye (for example, beta carotene or anthocyanin) without plating characteristics is added to an edible substrate, the dextrose appears white, grey and/or speckled.

The combination of silica and dye may produce a brighter color than the use of the dye alone. The L* value of the dye adsorbed onto the silica is at least about 5%, at least about 10%, at least about 15%, at least about 20%, or at least about 25% greater than the L* value of the dye before being adsorbed onto the silica, or of a comparable amount of dye without silica. Use of the colored powder may facilitate less dye being combined with an edible substrate to produce a desired color. For example, in a mixture or combination of edible particles and colored powder, the colored powder is suitably is present at less than about 5%, less than about 2%, less than about 1%, less than about 0.8%, less than about 0.6%, less than about 0.5% less than about 0.4%, less than about 0.3%, or less than about less than about 0.25% of the total weight of the edible particles and colored powder. For example a colored powder containing red cabbage and silica may be combined at about 0.2% by weight with 99.8% by weight dextrose to provide a magenta shade. A colored powder containing beta carotene and silica may be combined at about 0.8% by weight with 99.2% dextrose to provide a yellow shade.

The following examples are illustrative and are not to be construed as limiting the scope of the invention.

### Example 1

A colored powder was made by blending in a WARING® blender: 30 g of SIPERNAT® 50 (precipitated silica, commercially available from Evonik Degussa) with 20 g of a 2% water emulsion of beta carotene (Product No. 3030; commercially available from Sensient Technologies Corporation). The beta carotene was added drop-wise to the silica while blending on a low speed. 10.72 g of a 25% solution of ascorbic acid were then added drop-wise to the mixture blending on a low speed. The resulting powder had a good even yellow hue, and retained the yellow color and did not spoil after storage in sealed plastic jars at room temperature (about 68-72° F.) or under refrigeration (about 35-40° F.) for at least two weeks.

### Example 2

A colored powder was made by blending in a WARING® blender: 31.3 g of SIPERNAT® 50 S (synthetic amorphous precipitated silica, commercially available from Evonik Degussa) with 8.5 g of a 45% KOH solution, added drop-wise to the silica while blending on low speed. 7.02 g of a 50:50 blend of di-alpha tocopherol (commercially available from Roche, Switzerland) and DURKEX® (a partially hydrogenated soybean and cottonseed oil commercially available from Loders Croklaan, Netherlands) were then added drop-wise while blending on low speed. The tocopherol/DURKEX® combination was added drop-wise to the silica while blending on low speed. 49.2 g of a red cabbage solution (Product No. 3810; commercially available from Sensient Technologies Corporation) were then added drop-wise while blending on a low speed. The powder had an even blue color which turned green and retained the green color after storage overnight.

### Example 3

A colored powder was made by adding to a WARING® blender 20 g of SIPERNAT® 500 LS (synthetic amorphous precipitated silica, commercially available from Evonik Degussa). 33.43 g of a 10% aqueous solution of beta-carotene (a powder water-soluble form; a more concentrated version of Product No. 3044, commercially available from Sensient Technologies Corporation, Germany) commercially available from Sensient Technologies Corporation, Germany) were added slowly to the silica, drop-wise while blending on low speed. 11.49 g of ascorbic acid (commercially available from Roche, Switzerland) were gradually added while blending on low speed. The powder had an even light yellow color.

### Example 4

A colored powder was made by adding to a WARING® blender 20 g of SIPERNAT® 500 LS (synthetic amorphous precipitated silica, commercially available from Evonik Degussa). 2.27 g of a 2% water emulsion of beta carotene (Product No. 3030; commercially available from Sensient Technologies Corporation) were added slowly, drop-wise while blending on low speed. The powder had an even orange color.

### Example 5

A colored powder was made by adding to a WARING® blender 40 g of SIPERNAT® 22 (synthetic amorphous precipitated silica, commercially available from Evonik Degussa). 18.5 g of a 2% water emulsion of beta carotene (Product No. 3030; commercially available from Sensient Technologies Corporation) were added slowly to the silica, drop-wise while blending on low speed. 10 g of 22% aqueous solution of ascorbic acid (commercially available from Roche, Switzerland) were gradually added drop-wise while blending on low speed. The powder produced an even orange color when mixed with dextrose.

### Example 6

A colored powder was made by adding to a WARING® blender 40 g of SIPERNAT® 22 (synthetic amorphous precipitated silica, commercially available from Evonik Degussa). 23.84 g of a red cabbage solution (Product No. 3810; commercially available from Sensient Technologies Corporation) were then added drop-wise while blending on a low speed. 1.25 g of di-alpha tocopherol (commercially available from Roche, Switzerland) were then added drop-wise while blending on low speed. The powder had an even magenta color.

### Example 7

A colored powder was made by adding to a WARING® blender 40 g of SIPERNAT® 22 (synthetic amorphous precipitated silica, commercially available from Evonik Degussa). 5.17 g of a 45% KOH solution were added drop-wise to the silica while blending on low speed. 14.91 g of a red cabbage solution (Product No. 3810; commercially available from Sensient Technologies Corporation) were then added drop-wise while blending on a low speed. 1.05 g of di-alpha tocopherol (commercially available from Roche, Switzerland) were then added drop-wise while blending on low speed. The powder had an even purple color.

### Example 8

A colored powder was made by blending in a WARING® blender 40 g of SIPERNAT® 50 (precipitated silica, commercially available from Evonik Degussa) with 20 g of a 2% water emulsion of beta carotene (Product No. 3030; commercially available from Sensient Technologies Corporation). The beta carotene was added drop-wise to the silica while blending on a low speed. 10.33 g of a 25% solution of ascorbic acid were then added drop-wise to the mixture blending on a low speed. The resulting powder had a good even yellow hue when mixed with dextrose.

### Example 9

A colored powder was made by adding to a WARING® blender 40 g of SIPERNAT® 50 (precipitated silica, commercially available from Evonik Degussa). 21.29 g of a red cabbage solution (Product No. 3810; commercially available from Sensient Technologies Corporation) were added drop-wise to the silica while blending on a low speed. 5.01 g of a 50% solution of citric acid were then added drop-wise to the mixture blending on a low speed. 1.08 g of di-alpha tocopherol (commercially available from Roche, Switzerland) were then added drop-wise while blending on low speed. The resulting powder had a good even magenta hue.

### Example 10

A colored powder was made by adding to a WARING® blender 40 g of SIPERNAT® 50 (precipitated silica, commercially available from Evonik Degussa). 10.11 g of a 50% solution of citric acid were then added drop-wise to the mixture blending on a low speed. 21.07 g of a red cabbage solution (Product No. 3810; commercially available from Sensient Technologies Corporation) were then added drop-wise while blending on a low speed. 1.1 g of di-alpha tocopherol (commercially available from Roche, Switzerland) were then added drop-wise while blending on low speed. The resulting powder had a good even magenta color, which was retained after storage in sealed plastic jars at room temperature for at least 4 weeks.

### Example 11

A colored powder was made by adding to a WARING® blender 40 g of SIPERNAT® 50 (synthetic amorphous precipitated silica, commercially available from Evonik Degussa). 17.36 g of a 17% aqueous solution of caramel (absorbance of 0.1% sol at 610 nm between about 0.03 to 0.035) (commercially available from Sethness, Ill.) were added slowly, drop-wise while blending on low speed. The powder had an even light tan color, and retained color after storage in sealed plastic jars for 4 weeks.

### Example 12

A colored powder was made by adding to a WARING® blender 30 g of SIPERNAT® 50 (precipitated silica, commercially available from Evonik Degussa). 10.72 g of a 25% solution of ascorbic acid were then added drop-wise to the mixture blending on a low speed. 20 g of a 2% water emulsion of beta carotene (Product No. 3030; commercially available from Sensient Technologies Corporation) were added drop-wise while blending on a low speed. The resulting powder had a good even yellow hue, and retained color after storage in sealed plastic jars for at least 1 week at about 22° C.

The L*, a*, b*, DE*, strength and DEcmc values during storage at about 22° C. are shown in Table 1.

TABLE 1

| Days in | | | | | Weight | |
|---|---|---|---|---|---|---|
| Storage | L* | a* | b* | DE* | Strength | DEcmc |
| 0 | 79.16 | 20.66 | 53.05 | | | |
| 1 | 79.87 | 16.95 | 48.54 | 5.89 | 80.11 | 2.57 |
| 3 | 80.28 | 15.14 | 46.35 | 8.75 | 71.72 | 3.85 |
| 7 | 80.28 | 14.52 | 45.18 | 10.05 | 68.66 | 4.33 |
| 9 | 80.37 | 13.75 | 43.17 | 12.12 | 63.39 | 5.02 |
| 10 | 81.06 | 13.26 | 42.38 | 13.13 | 58.58 | 5.42 |
| 13 | 81.38 | 12.29 | 39.71 | 15.9 | 51.82 | 6.36 |
| 15 | 81.12 | 12.21 | 39.62 | 15.99 | 52.66 | 6.4 |
| 17 | 81.11 | 12.01 | 39.28 | 16.37 | 52.03 | 6.56 |

### Example 13

A colored powder was made by adding to a WARING® blender 40 g of SIPERNAT® 50 (precipitated silica, commercially available from Evonik Degussa). 21.29 g of a red cabbage solution (Product No. 3810; commercially available from Sensient Technologies Corporation) were then added drop-wise while blending on a low speed. 5.01 g of a 50% solution of citric acid were then added drop-wise to the mixture blending on a low speed. 1.08 g of di-alpha tocopherol (commercially available from Roche, Switzerland) were then added drop-wise while blending on low speed. The resulting powder had a good even magenta color, which was retained after storage at about 22° C. in sealed plastic jars for at least 2 weeks.

The L*, a*, b*, DE*, strength and DEcmc values of the powder during storage at 22° C. [correct] are shown in Table 2.

TABLE 2

| Days in | | | | | Weight | |
|---|---|---|---|---|---|---|
| Storage | L* | a* | b* | DE* | Strength | DEcmc |
| 0 | 52.92 | 34.01 | −6.6 | | | |
| 1 | 51.04 | 32.82 | −6.3 | 2.24 | 113.08 | 1.01 |
| 3 | 51.24 | 32.57 | −6.73 | 2.21 | 110.6 | 1.02 |
| 7 | 51.25 | 32.39 | −6.89 | 2.35 | 110.17 | 1.1 |
| 9 | 50.96 | 32.07 | −6.92 | 2.77 | 112.03 | 1.3 |
| 10 | 50.97 | 32.03 | −6.9 | 2.79 | 111.92 | 1.31 |
| 13 | 50.82 | 31.72 | −6.91 | 3.11 | 112.73 | 1.46 |
| 15 | 50.8 | 31.73 | −7.07 | 3.15 | 112.69 | 1.49 |
| 17 | 50.24 | 31.56 | −6.77 | 3.63 | 117.38 | 1.67 |
| 20 | 50.87 | 31.54 | −7.09 | 3.24 | 111.94 | 1.54 |
| 24 | 50.91 | 31.45 | −7.24 | 3.31 | 111.32 | 1.6 |
| 27 | 50.46 | 31.31 | −7 | 3.67 | 115.03 | 1.72 |
| 31 | 50.41 | 31.3 | −6.86 | 3.7 | 115.63 | 1.72 |
| 35 | 50.05 | 31.08 | −6.75 | 4.1 | 118.48 | 1.89 |
| 50 | 50.27 | 30.89 | −6.97 | 4.11 | 116.18 | 1.93 |
| 67 | 50.36 | 30.46 | −6.91 | 4.39 | 115.23 | 20.6 |

### Example 14

A colored powder was made by adding to a WARING® blender 30 g of SIPERNAT® 50 (precipitated silica, commercially available from Evonik Degussa). 21.32 g of 1.2% betanin solution from beets (a more concentrated form of Product No. 3613; commercially available from Sensient Technologies Corporation) were then added drop-wise while blending on a low speed. 4.8 g of a 25% solution of ascorbic acid were then added drop-wise to the mixture blending on a low speed. 5.17 g of 25% sodium hexametaphosphate solution (commercially available from Solutia, St. Louis) were then added drop-wise while blending on low speed. 7.25 g of a 10% solution of EDTA were then added drop-wise while blending on low speed. The resulting powder had a good even magenta color, which was retained after storage in sealed plastic jars at about 22° C. for at least 1 week.

The L*, a*, b*, DE*, strength and DEcmc values of the powder during storage at room temperature are shown in Table 3.

TABLE 3

| Days in | | | | | Weight | |
|---|---|---|---|---|---|---|
| Storage | L* | a* | b* | DE* | Strength | DEcmc |
| 0 | 60.47 | 30.54 | −5.64 | | | |
| 1 | 63.31 | 27.62 | −2.47 | 5.17 | 83.46 | 2.73 |
| 2 | 64.69 | 25.18 | 1.3 | 9.73 | 78.99 | 5.54 |
| 3 | 68.09 | 22.99 | 3.28 | 13.96 | 62.48 | 7.68 |
| 7 | 73.93 | 16.42 | 8 | 23.8 | 41.78 | 13.1 |

### Example 15

A colored powder was made by adding to a WARING® blender 30 g of SIPERNAT® 50 (precipitated silica, commercially available from Evonik Degussa). 1.5 g of a 45% solution of potassium hydroxide were then added drop-wise to the mixture blending on a low speed. 22.26 g of a red cabbage solution (Product No. 3810; commercially available from Sensient Technologies Corporation) were then added drop-wise while blending on a low speed. 5 g of 25% sodium hexametaphosphate solution (commercially available from Solutia, St. Louis) were then added drop-wise while blending on low speed. The resulting powder had a blue color which shifted to a purple color. The purple color was retained after storage in sealed plastic jars at 22° C. for at least 2 weeks.

The L*, a*, b*, DE*, strength and DEcmc values of the powder during storage at 22° C. are shown in Table 4.

TABLE 4

| Days in | | | | | Weight | |
|---|---|---|---|---|---|---|
| Storage | L* | a* | b* | DE* | Strength | DEcmc |
| 0 | 54.17 | 11.19 | −16.32 | | | |
| 1 | 53.78 | 7.74 | −17.5 | 3.67 | 103.86 | 3.45 |
| 3 | 53.96 | 7.01 | −16.84 | 4.22 | 103.42 | 3.86 |
| 7 | 54.43 | 6.82 | −16.48 | 4.39 | 100.45 | 3.93 |
| 9 | 56.6 | 6.28 | −14.52 | 5.77 | 87.45 | 4.2 |
| 10 | 57.3 | 6.2 | −14.09 | 6.3 | 83.48 | 4.33 |
| 13 | 58.21 | 6.05 | −11.69 | 8.02 | 79.7 | 4.81 |
| 15 | 59.24 | 5.84 | −10.72 | 9.25 | 74.68 | 5.35 |
| 17 | 59.83 | 5.79 | −9.97 | 10.07 | 72.01 | 5.72 |
| 20 | 60.49 | 5.92 | −8.71 | 11.21 | 69.42 | 6.29 |

### Example 16

A colored powder was made by adding to a WARING® blender 30 g of SIPERNAT® 50 (precipitated silica, commercially available from Evonik Degussa). 3 g of a 45% solution of potassium hydroxide were then added drop-wise to the mixture blending on a low speed. 22.85 g of a red cabbage solution (Product No. 3810; commercially available from Sensient Technologies Corporation) were then added drop-wise while blending on a low speed. 5 g of 25% sodium hexametaphosphate solution (commercially available from Solutia, St. Louis) were then added drop-wise while blending on low speed. The resulting powder had a blue color, which was retained after storage in sealed plastic jars at 22° C. for at least 1 day.

### Example 17

A colored powder was made by adding to a WARING® blender 30 g of SIPERNAT® 50 (precipitated silica, commercially available from Evonik Degussa). 5 g of a 45% solution of potassium hydroxide were then added drop-wise to the mixture blending on a low speed. 22.34 g of a E1=8-10% black carrot anthocyanin solution (Product No. 3847; commercially available from Sensient Technologies Corporation) were then added drop-wise while blending on a low speed. 5 g of 25% sodium hexametaphosphate solution (commercially available from Solutia, St. Louis) were then added drop-wise while blending on low speed. The resulting powder had a purple color, which was retained after storage in sealed plastic jars at 22° C. for at least 2 weeks.

The L*, a*, b*, DE*, strength and DEcmc values of the powder during storage at 22° C. are shown in Table 5.

TABLE 5

| Days in | | | | | Weight | |
|---|---|---|---|---|---|---|
| Storage | L* | a* | b* | DE* | Strength | DEcmc |
| 0 | 57.1 | 13.01 | −8.74 | | | |
| 1 | 57.2 | 12.11 | −9.26 | 1.05 | 98.79 | 0.97 |
| 5 | 58.24 | 11.19 | −9.13 | 2.18 | 92.14 | 1.66 |
| 11 | 58.09 | 11.17 | −8.98 | 2.1 | 93.22 | 1.6 |
| 13 | 58.79 | 10.82 | −8.4 | 2.79 | 89.46 | 1.81 |
| 15 | 58.63 | 10.82 | −8.11 | 2.75 | 90.82 | 1.77 |
| 18 | 59.1 | 10.7 | −7.97 | 3.15 | 88.04 | 1.93 |
| 22 | 59.48 | 10.47 | −7.77 | 3.61 | 85.89 | 2.16 |
| 25 | 59.98 | 10.37 | −7.57 | 4.08 | 83.06 | 2.35 |
| 29 | 60.18 | 10.35 | −7.31 | 4.31 | 82.18 | 2.46 |
| 33 | 59.65 | 10.32 | −6.82 | 4.18 | 85.9 | 2.51 |

The L*, a*, b*, DE*, strength and DEcmc values of the powder during refrigerated storage are shown in Table 6.

TABLE 6

| Days in | | | | | Weight | |
|---|---|---|---|---|---|---|
| Storage | L* | a* | b* | DE* | Strength | DEcmc |
| 0 | 57.1 | 13.01 | −8.74 | | | |
| 1 | 57.19 | 12.38 | −9.51 | 1 | 98.61 | 0.96 |
| 5 | 57.02 | 12 | −9.62 | 1.34 | 99.71 | 1.28 |
| 11 | 57.06 | 11.85 | −9.89 | 1.63 | 99.2 | 1.57 |
| 13 | 57.6 | 11.61 | −9.35 | 1.6 | 96.03 | 1.4 |
| 15 | 56.65 | 11.75 | −9.74 | 1.66 | 102.29 | 1.54 |
| 18 | 57.48 | 11.52 | −9.45 | 1.69 | 96.76 | 1.52 |
| 22 | 57.72 | 11.45 | −9.47 | 1.83 | 95.14 | 1.59 |
| 25 | 57.59 | 11.52 | −9.54 | 1.76 | 96 | 1.57 |
| 29 | 58.18 | 11.31 | −9.41 | 2.12 | 92.21 | 1.7 |
| 33 | 57.37 | 11.25 | −9.21 | 1.84 | 97.84 | 1.58 |
| 48 | 57.61 | 11.33 | −9.19 | 1.81 | 96.21 | 1.52 |
| 75 | 58.1 | 11.19 | −9.21 | 2.13 | 93.01 | 1.68 |

### Example 18

A colored powder was made by adding to a WARING® blender 30 g of SIPERNAT® 50 (precipitated silica, commercially available from Evonik Degussa). 20 g of a 2% water emulsion of beta carotene (Product No. 3030; commercially available from Sensient Technologies Corporation) were added drop-wise while blending on a low speed. 12 g of a 25% solution of ascorbic acid were then added drop-wise to the mixture blending on a low speed. 2.62 g of di-alpha tocopherol (commercially available from Roche, Switzerland) were then added drop-wise while blending on low speed. The resulting powder had a good yellow color, which was retained after storage in sealed plastic jars at room temperature for 1 week.

The L*, a*, b*, DE*, strength and DEcmc values of the powder during storage at room temperature are shown in Table 7.

TABLE 7

| Days in | | | | | Weight | |
|---|---|---|---|---|---|---|
| Storage | L* | a* | b* | DE* | Strength | DEcmc |
| 0 | 79.33 | 20.13 | 56.23 | | | |
| 1 | 78.98 | 19.21 | 52.62 | 3.74 | 89.53 | 1.37 |
| 5 | 78.86 | 18.6 | 50.45 | 6 | 83.33 | 2.19 |
| 8 | 78.82 | 18.58 | 50.06 | 6.38 | 82.39 | 2.34 |
| 11 | 78.99 | 18.29 | 49.78 | 6.71 | 80.55 | 2.44 |
| 13 | 79.27 | 17.95 | 49.68 | 6.9 | 78.71 | 2.49 |
| 15 | 78.75 | 18.8 | 50.56 | 5.85 | 84.28 | 2.16 |
| 18 | 79.53 | 17.76 | 49.38 | 7.25 | 76.45 | 2.61 |
| 22 | 78.84 | 17.12 | 48.28 | 8.52 | 77.09 | 3.07 |
| 25 | 78.3 | 16.12 | 46.88 | 10.23 | 76.1 | 3.71 |
| 29 | 78.62 | 15.59 | 45.54 | 11.63 | 70.93 | 4.22 |
| 33 | 78.04 | 15.28 | 44.49 | 12.76 | 71.1 | 4.62 |
| 48 | 78 | 15.3 | 42.25 | 14.85 | 65.9 | 5.35 |

The L*, a*, b*, DE*, strength and DEcmc values of the powder during refrigerated storage are shown in Table 8.

TABLE 8

| Days in | | | | | Weight | |
|---|---|---|---|---|---|---|
| Storage | L* | a* | b* | DE* | Strength | DEcmc |
| 0 | 79.33 | 20.13 | 56.23 | | | |
| 1 | 79.03 | 19.88 | 53.69 | 2.57 | 92.86 | 1.02 |
| 5 | 78.06 | 19.93 | 52.02 | 4.4 | 93.29 | 1.81 |
| 8 | 79.05 | 18.97 | 51.26 | 5.11 | 84.75 | 1.89 |
| 11 | 78.53 | 19.13 | 50.92 | 5.46 | 86.78 | 2.06 |
| 13 | 78.43 | 19.27 | 50.74 | 5.63 | 86.74 | 2.16 |
| 15 | 79.02 | 17.99 | 49.71 | 6.87 | 80.11 | 2.48 |
| 18 | 79.12 | 18.87 | 50.1 | 6.26 | 80.8 | 2.34 |
| 22 | 78.67 | 18.97 | 50.19 | 6.19 | 83.64 | 2.33 |
| 25 | 78.68 | 18.94 | 50 | 6.38 | 83.02 | 2.41 |
| 29 | 79.11 | 18.76 | 49.88 | 6.5 | 80.19 | 2.42 |
| 33 | 78.55 | 18.99 | 49.97 | 6.41 | 83.66 | 2.43 |
| 48 | 78.48 | 18.55 | 49.17 | 7.28 | 81.67 | 2.7 |
| 75 | 78.6 | 18.58 | 49.14 | 7.3 | 80.9 | 2.72 |

### Example 19

A colored powder was made by adding to a WARING® blender 30 g of SIPERNAT® 50 (precipitated silica, commercially available from Evonik Degussa). 10.8 g of a 2% water emulsion of beta carotene (Product No. 3030; commercially available from Sensient Technologies Corporation) were added drop-wise while blending on a low speed. 11.83 g of 1.2% betanin solution (from beets; commercially available from Sensient Technologies Corporation) were then added drop-wise while blending on a low speed. 5 g of 25% sodium hexametaphosphate solution (commercially available from Solutia, St. Louis) were then added drop-wise while blending on low speed. The resulting powder had a rusty color, which turned slightly yellow over time.

The L*, a*, b*, DE*, strength and DEcmc values of the powder during storage at room temperature are shown in Table 9.

TABLE 9

| Days in | | | | | Weight | |
|---|---|---|---|---|---|---|
| Storage | L* | a* | b* | DE* | Strength | DEcmc |
| 0 | 62.37 | 25.68 | 23.62 | | | |
| 1 | 65.42 | 21.99 | 25.8 | 5.26 | 84.87 | 4.52 |
| 5 | 73.56 | 14.93 | 31.53 | 17.42 | 55.41 | 14.56 |
| 9 | 75.17 | 13.2 | 32.25 | 19.84 | 50.26 | 16.55 |

The L*, a*, b*, DE*, strength and DEcmc values of the powder during refrigerated storage are shown in Table 10.

TABLE 10

| Days in | | | | | Weight | |
|---|---|---|---|---|---|---|
| Storage | L* | a* | b* | DE* | Strength | DEcmc |
| 0 | 62.37 | 25.68 | 23.62 | | | |
| 1 | 62.86 | 24.27 | 24.36 | 1.66 | 98.19 | 1.59 |
| 5 | 64.57 | 22.6 | 25.44 | 4.2 | 89.31 | 3.73 |
| 7 | 64.85 | 22.5 | 26.14 | 4.76 | 89.23 | 4.3 |
| 13 | 67.17 | 20.22 | 26.17 | 7.71 | 75.53 | 6.33 |
| 15 | 68.28 | 19.52 | 26.97 | 9.17 | 71.28 | 7.5 |
| 18 | 68.86 | 19.1 | 27.04 | 9.85 | 68.5 | 7.95 |

### Example 20

A colored powder was made by adding to a WARING® blender 30 g of SIPERNAT® 50 (precipitated silica, commercially available from Evonik Degussa). 5.2 g of a 2% water emulsion of beta carotene (Product No. 3030; commercially available from Sensient Technologies Corporation) were added drop-wise while blending on a low speed. 5 g of 25% ascorbic acid solution were then added drop-wise while blending on low speed. The resulting powder had a yellow color, which was retained after storage in sealed plastic jars at room temperature and under refrigeration for 2 days.

The L*, a*, b*, DE*, strength and DEcmc values of the powder during storage at room temperature are shown in Table 11.

TABLE 11

| Days in | | | | | Weight | |
|---|---|---|---|---|---|---|
| Storage | L* | a* | b* | DE* | Strength | DEcmc |
| 0 | 82.16 | 15.58 | 60.25 | | | |
| 3 | 84.64 | 7.2 | 42.4 | 19.88 | 42.39 | 7.53 |
| 5 | 86.54 | 5.32 | 32.32 | 30.08 | 24.33 | 10.94 |

The L*, a*, b*, DE*, strength and DEcmc values of the powder during refrigerated storage are shown in Table 12.

TABLE 12

| Days in | | | | | Weight | |
|---|---|---|---|---|---|---|
| Storage | L* | a* | b* | DE* | Strength | DEcmc |
| 0 | 82.16 | 15.58 | 60.25 | | | |
| 3 | 82.61 | 12.47 | 56.83 | 4.65 | 84.97 | 2.14 |
| 5 | 83.33 | 9.91 | 53.45 | 8.94 | 71 | 4.02 |
| 7 | 84.34 | 7.4 | 48.91 | 14.16 | 55.6 | 6.11 |
| 10 | 84.75 | 6.12 | 44.52 | 18.54 | 45.59 | 7.57 |

### Example 21

A colored powder was made by adding to a WARING® blender 30 g of SIPERNAT® 50 (precipitated silica, commercially available from Evonik Degussa). 10.22 g of a 2% water emulsion of beta carotene (Product No. 3030; commercially available from Sensient Technologies Corporation) were added drop-wise while blending on a low speed. 5 g of 25% ascorbic acid solution were then added drop-wise while blending on low speed. The resulting powder had a yellow color, which was retained after storage in sealed plastic jars at room temperature for 1 week and refrigeration for 2 weeks.

The L*, a*, b*, DE*, strength and DEcmc values of the powder during storage at room temperature are shown in Table 13.

TABLE 13

| Days in | | | | | Weight | |
|---|---|---|---|---|---|---|
| Storage | L* | a* | b* | DE* | Strength | DEcmc |
| 0 | 81.3 | 16.59 | 64.21 | | | |
| 3 | 83.64 | 10.6 | 58.19 | 8.81 | 67.53 | 4.08 |
| 5 | 84.78 | 7.9 | 51.8 | 15.54 | 48.9 | 6.45 |
| 7 | 85.43 | 5.61 | 43.92 | 23.44 | 34.46 | 9.05 |
| 10 | 88.11 | −0.83 | 24.34 | 44.04 | 12.44 | 16.67 |

The L*, a*, b*, DE*, strength and DEcmc values of the powder during refrigerated storage are shown in Table 14.

TABLE 14

| Days in | | | | | Weight | |
|---|---|---|---|---|---|---|
| Storage | L* | a* | b* | DE* | Strength | DEcmc |
| 0 | 81.3 | 16.59 | 64.21 | | | |
| 3 | 81.95 | 15 | 64.32 | 1.71 | 95.94 | 1.11 |
| 5 | 82.72 | 13.47 | 63.5 | 3.49 | 88.14 | 2.1 |
| 7 | 83.63 | 11.77 | 62.28 | 5.68 | 79.04 | 3.23 |
| 10 | 84.36 | 10.75 | 61.2 | 7.24 | 72.11 | 3.93 |
| 14 | 84.69 | 9.23 | 58.96 | 9.65 | 64.67 | 4.96 |
| 17 | 84.83 | 8.44 | 57.34 | 11.22 | 60.19 | 5.54 |
| 21 | 85.85 | 7.1 | 54.58 | 14.26 | 50.44 | 6.65 |

### Example 22

A colored powder was made by adding to a WARING® blender 30 g of SIPERNAT® 50 (precipitated silica, commercially available from Evonik Degussa). 5.5 g of a 2% water emulsion of beta carotene (Product No. 3030; commercially available from Sensient Technologies Corporation) were added drop-wise while blending on a low speed. 10 g of 25% ascorbic acid solution were then added drop-wise while blending on low speed. 3 g of di-alpha tocopherol (commercially available from Roche, Switzerland) were then added drop-wise while blending on low speed. The resulting powder had a yellow color, which was retained after storage in sealed plastic jars at room temperature for 1 week and refrigeration for 2 weeks.

The L*, a*, b*, DE*, strength and DEcmc values of the powder during storage at room temperature are shown in Table 15.

TABLE 15

| Days in | | | | | Weight | |
|---|---|---|---|---|---|---|
| Storage | L* | a* | b* | DE* | Strength | DEcmc |
| 0 | 84.68 | 12.38 | 55.91 | | | |
| 3 | 85.17 | 10.56 | 53.46 | 3.09 | 87.82 | 1.38 |
| 5 | 85.7 | 9.12 | 50.01 | 6.81 | 73.86 | 2.79 |
| 7 | 86.28 | 7.87 | 47.02 | 10.09 | 62.87 | 4.06 |
| 10 | 87.3 | 6.35 | 41.77 | 15.59 | 46.97 | 6.07 |

The L*, a*, b*, DE*, strength and DEcmc values of the powder during refrigerated storage are shown in Table 16.

TABLE 16

| Days in | | | | | Weight | |
|---|---|---|---|---|---|---|
| Storage | L* | a* | b* | DE* | Strength | DEcmc |
| 0 | 84.68 | 12.38 | 55.91 | | | |
| 3 | 84.62 | 11.8 | 55.81 | 0.6 | 99.93 | 0.4 |
| 5 | 84.76 | 11.06 | 55 | 1.6 | 95.86 | 0.9 |
| 7 | 85.14 | 10.1 | 53.73 | 3.19 | 88.78 | 1.61 |
| 10 | 85.23 | 9.79 | 53.17 | 3.81 | 86.35 | 1.86 |
| 14 | 85.28 | 8.74 | 51.1 | 6.05 | 79.37 | 2.75 |
| 17 | 85.59 | 8.04 | 49.51 | 7.79 | 72.91 | 3.41 |
| 21 | 85.96 | 7.54 | 48.45 | 8.98 | 67.99 | 3.89 |
| 25 | 86.11 | 6.76 | 46.35 | 11.18 | 61.88 | 4.71 |
| 39 | 86.95 | 5.18 | 41.22 | 16.52 | 47.14 | 6.67 |

### Example 23

A colored powder was made by adding to a WARING® blender 30 g of SIPERNAT® 50 (precipitated silica, commercially available from Evonik Degussa). 10.15 g of a 2% water emulsion of beta carotene (Product No. 3030; commercially available from Sensient Technologies Corporation) were added drop-wise while blending on a low speed. 10 g of 25% ascorbic acid solution were then added drop-wise while blending on low speed. 3.3 g of di-alpha tocopherol (commercially available from Roche, Switzerland) were then added drop-wise while blending on low speed. The resulting powder had a yellow color, which was retained after storage in sealed plastic jars at room temperature for 1 week and refrigeration for 2 weeks.

The L*, a*, b*, DE*, strength and DEcmc values of the powder during storage at room temperature are shown in Table 17.

TABLE 17

| Days in | | | | | Weight | |
|---|---|---|---|---|---|---|
| Storage | L* | a* | b* | DE* | Strength | DEcmc |
| 0 | 82.09 | 16.84 | 56.65 | | | |
| 3 | 82.6 | 14.13 | 51.5 | 5.84 | 79.36 | 2.25 |
| 5 | 82.87 | 13.35 | 49.45 | 8.04 | 72.04 | 3.05 |
| 7 | 82.75 | 13.04 | 48.8 | 8.74 | 70.89 | 3.31 |
| 10 | 83.27 | 12.38 | 46.71 | 10.96 | 63.15 | 4.1 |
| 14 | 82.86 | 11.74 | 45.09 | 12.66 | 61.2 | 4.73 |
| 17 | 82.86 | 10.71 | 42.75 | 15.21 | 56.02 | 5.69 |
| 21 | 83.07 | 8.91 | 39.4 | 19.01 | 48.55 | 7.2 |

The L*, a*, b*, DE*, strength and DEcmc values of the powder during refrigerated storage are shown in Table 18.

TABLE 18

| Days in | | | | | Weight | |
|---|---|---|---|---|---|---|
| Storage | L* | a* | b* | DE* | Strength | DEcmc |
| 0 | 82.09 | 16.84 | 56.65 | | | |
| 3 | 81.81 | 17.47 | 56.07 | 0.9 | 99.83 | 0.6 |
| 5 | 81.78 | 17.92 | 53.79 | 3.07 | 92 | 1.66 |
| 7 | 81.91 | 17.74 | 52.02 | 4.72 | 85.33 | 2.25 |
| 10 | 82.34 | 17.84 | 51.11 | 5.64 | 80.16 | 2.67 |
| 14 | 81.91 | 17.92 | 50.85 | 5.9 | 81.83 | 2.81 |
| 17 | 82.25 | 17.63 | 49.99 | 6.7 | 77.38 | 3.03 |
| 21 | 82.26 | 17.91 | 50.73 | 6.01 | 79.46 | 2.85 |
| 25 | 81.89 | 17.93 | 50.38 | 6.37 | 80.52 | 3.01 |
| 40 | 81.88 | 17.62 | 48.84 | 7.85 | 76.13 | 3.51 |
| 67 | 81.67 | 17.58 | 47.37 | 9.32 | 73.25 | 4.11 |

### Example 24

A colored powder was made by adding to a WARING® blender 30 g of SIPERNAT® 50 (precipitated silica, commercially available from Evonik Degussa). 15.45 g of 1.2% betanin solution from beets (a more concentrated form of Product No. 3613; commercially available from Sensient Technologies Corporation) were added drop-wise while blending on a low speed. 3.3 g of 25% ascorbic acid solution were then added drop-wise while blending on low speed. 5 g of 25% sodium hexametaphosphate solution (commercially available from Solutia, St. Louis) were then added drop-wise while blending on low speed. The resulting powder had a pink/red color, which was retained after storage at room temperature for 2 days and refrigeration for 2 weeks.

The L*, a*, b*, DE*, strength and DEcmc values of the powder during storage at room temperature are shown in Table 19.

TABLE 19

| Days in | | | | | Weight | |
|---|---|---|---|---|---|---|
| Storage | L* | a* | b* | DE* | Strength | DEcmc |
| 0 | 60.65 | 30.88 | −7.15 | | | |
| 3 | 65.06 | 25.95 | −2.57 | 8.05 | 74.52 | 4.09 |
| 5 | 69.27 | 21.44 | 1.53 | 15.46 | 56.2 | 7.98 |
| 7 | 71.84 | 18.54 | 4.08 | 20.09 | 47.3 | 10.52 |

The L*, a*, b*, DE*, strength and DEcmc values of the powder during refrigerated storage are shown in Table 20.

TABLE 20

| Days in | | | | | Weight | |
|---|---|---|---|---|---|---|
| Storage | L* | a* | b* | DE* | Strength | DEcmc |
| 0 | 60.65 | 30.88 | −7.15 | | | |
| 3 | 60.98 | 30.2 | −6.99 | 0.78 | 97.25 | 0.37 |
| 5 | 61.81 | 29.49 | −6.49 | 1.93 | 91.73 | 0.9 |
| 7 | 60.82 | 29.68 | −6.39 | 1.44 | 98.94 | 0.73 |
| 10 | 62.79 | 28.47 | −4.82 | 3.99 | 86.74 | 2.03 |
| 14 | 64.02 | 27.29 | −4.2 | 5.74 | 79.29 | 2.83 |
| 17 | 64.29 | 26.63 | −2.93 | 7.01 | 78.88 | 3.62 |
| 21 | 65.69 | 25.78 | −1.85 | 8.92 | 71.82 | 4.6 |
| 25 | 65.97 | 24.86 | −0.65 | 10.34 | 71.35 | 5.46 |
| 40 | 67.84 | 22.66 | 1.85 | 14.16 | 63.83 | 7.6 |
| 67 | 71.39 | 18.91 | 5.4 | 20.41 | 50.68 | 11.06 |

### Example 25

A colored powder was made by adding to a WARING® blender 30 g of SIPERNAT® 50 (precipitated silica, commercially available from Evonik Degussa). 3.3 g of a 10% tetrasodium pyrophosphate solution were added drop-wise while blending on low speed. 20.95 g of a red cabbage solution (Product No. 3810; commercially available from Sensient Technologies Corporation) were then added drop-wise while blending on a low speed. The resulting powder had a purple color, which was retained after storage in sealed plastic jars at room temperature and under refrigeration for at least 5 weeks.

The L*, a*, b*, DE*, strength and DEcmc values of the powder during storage at room temperature are shown in Table 21.

TABLE 21

| Days in | | | | | Weight | |
|---|---|---|---|---|---|---|
| Storage | L* | a* | b* | DE* | Strength | DEcmc |
| 0 | 51.46 | 24.27 | −13.16 | | | |
| 3 | 50.94 | 22.75 | −13.41 | 1.63 | 102.81 | 0.97 |
| 5 | 51.42 | 23.08 | −13.26 | 1.2 | 99.66 | 0.71 |
| 7 | 51.35 | 22.91 | −13.19 | 1.37 | 100.11 | 0.79 |
| 10 | 52.28 | 22.83 | −13.22 | 1.66 | 93.91 | 0.92 |
| 14 | 51.55 | 22.84 | −13.01 | 1.44 | 98.97 | 0.8 |
| 17 | 51.37 | 22.92 | −12.88 | 1.39 | 100.37 | 0.75 |
| 21 | 51.8 | 22.81 | −12.95 | 1.51 | 97.37 | 0.82 |
| 25 | 51.48 | 22.69 | −12.75 | 1.63 | 99.67 | 0.87 |
| 40 | 52.09 | 22.62 | −12.48 | 1.9 | 95.89 | 0.97 |
| 67 | 51.76 | 22.79 | −11.86 | 1.99 | 98.87 | 1.05 |

The L*, a*, b*, DE*, strength and DEcmc values of the powder during refrigerated storage are shown in Table 22.

TABLE 22

| Days in | | | | | Weight | |
|---|---|---|---|---|---|---|
| Storage | L* | a* | b* | DE* | Strength | DEcmc |
| 0 | 51.46 | 24.27 | −13.16 | | | |
| 3 | 50.7 | 23.36 | −13.93 | 1.42 | 104.22 | 0.91 |
| 5 | 51.18 | 23.11 | −13.84 | 1.38 | 100.84 | 0.92 |
| 7 | 51.35 | 22.88 | −13.68 | 1.49 | 99.71 | 0.96 |
| 10 | 51.46 | 23.03 | −13.55 | 1.3 | 99.1 | 0.83 |
| 14 | 51.35 | 22.51 | −13.66 | 1.83 | 99.56 | 1.16 |
| 17 | 50.67 | 22.79 | −13.92 | 1.84 | 104.14 | 1.17 |
| 21 | 51.63 | 22.79 | −13.85 | 1.64 | 97.59 | 1.09 |
| 25 | 50.64 | 22.55 | −13.73 | 1.99 | 104.41 | 1.23 |
| 40 | 51.26 | 22.69 | −13.43 | 1.62 | 100.46 | 0.99 |
| 67 | 51.35 | 22.51 | −13.66 | 1.83 | 99.56 | 1.16 |

### Example 26

A colored powder was made by adding to a WARING® blender 30 g of SIPERNAT® 50 (precipitated silica, commercially available from Evonik Degussa). 7 g of a 10% tetrasodium pyrophosphate solution were added drop-wise while blending on low speed. 19.06 g of a red cabbage solution (Product No. 3810; commercially available from Sensient Technologies Corporation) were then added drop-wise while blending on a low speed. 1.54 g of a 45% solution of potassium hydroxide were then added drop-wise to the mixture blending on a low speed. The resulting powder had a purple color, which was retained after refrigerated storage in sealed plastic jars for 1 week.

The L*, a*, b*, DE*, strength and DEcmc values of the powder during refrigerated storage are shown in Table 23.

TABLE 23

| Days in | | | | | Weight | |
|---|---|---|---|---|---|---|
| Storage | L* | a* | b* | DE* | Strength | DEcmc |
| 0 | 52.66 | 0.21 | −18.48 | | | |
| 3 | 54.38 | −0.89 | −16.59 | 2.78 | 90.48 | 1.84 |
| 5 | 58.3 | −2.97 | −11.04 | 9.86 | 72.29 | 6.58 |
| 7 | 60.15 | −3.12 | −7.8 | 13.46 | 64.97 | 8.79 |

### Example 27

A colored powder was made by adding to a WARING® blender 30 g of SIPERNAT® 50 (precipitated silica, commercially available from Evonik Degussa). 7.3 g of a 2% water emulsion of beta carotene (Product No. 3030; commercially available from Sensient Technologies Corporation) were added drop-wise while blending on a low speed. 25 g of 25% ascorbic acid solution were then added drop-wise while blending on low speed. 3 g of di-alpha tocopherol (commercially available from Roche, Switzerland) were then added drop-wise while blending on low speed. The resulting powder had a yellow color, which was retained after storage in sealed plastic jars at room temperature for 2 days and under refrigeration for 1 week.

The L*, a*, b*, DE*, strength and DEcmc values of the powder during storage at room temperature are shown in Table 24.

TABLE 24

| Days in | | | | | Weight | |
|---|---|---|---|---|---|---|
| Storage | L* | a* | b* | DE* | Strength | DEcmc |
| 0 | 83.38 | 14.05 | 58.15 | | | |
| 1 | 84.48 | 12.52 | 56.29 | 2.65 | 86.28 | 1.16 |
| 2 | 84.24 | 11.76 | 55.03 | 3.96 | 83.53 | 1.71 |
| 4 | 84.84 | 10.21 | 51.52 | 7.8 | 69.91 | 3.15 |
| 7 | 85.93 | 8.58 | 46.59 | 13.03 | 53.29 | 5.02 |

The L*, a*, b*, DE*, strength and DEcmc values of the powder during refrigerated storage are shown in Table 25.

TABLE 25

| Days in | | | | | Weight | |
|---|---|---|---|---|---|---|
| Storage | L* | a* | b* | DE* | Strength | DEcmc |
| 0 | 83.38 | 14.05 | 58.15 | | | |
| 1 | 83.74 | 14.23 | 59.09 | 1.02 | 101.11 | 0.37 |
| 2 | 83.85 | 13.31 | 57.96 | 0.89 | 96.11 | 0.52 |
| 4 | 84.17 | 12.56 | 57.04 | 2.02 | 90.68 | 1.04 |
| 7 | 84.51 | 12.2 | 56.32 | 2.83 | 86.13 | 1.33 |
| 11 | 84.65 | 11.45 | 54.11 | 4.97 | 78.31 | 2.05 |
| 14 | 84.5 | 11.35 | 53.62 | 5.39 | 77.67 | 2.18 |
| 18 | 84.71 | 10.96 | 52.78 | 6.33 | 74.01 | 2.54 |
| 22 | 84.7 | 10.56 | 51.29 | 7.81 | 69.95 | 3.04 |
| 37 | 85.37 | 8.99 | 45.87 | 13.43 | 53.95 | 5.04 |

### Example 28

A colored powder was made by adding to a WARING® blender 30 g of SIPERNAT® 50 (precipitated silica, commercially available from Evonik Degussa). 15 g of a red cabbage solution (Product No. 3810; commercially available from Sensient Technologies Corporation) were added drop-wise while blending on a low speed. 10 g of a 10% tetrasodium pyrophosphate solution were then added drop-wise while blending on low speed. 4.7 g of a 40 % solution of Gum arabic were then added drop-wise to the mixture blending on a low speed. The resulting powder had a purple color, which was retained after storage in sealed plastic jars at room temperature for 1 week and under refrigeration for 2 weeks

The L*, a*, b*, DE*, strength and DEcmc values of the powder during storage at room temperature are shown in Table 26.

TABLE 26

| Days in | | | | | Weight | |
|---|---|---|---|---|---|---|
| Storage | L* | a* | b* | DE* | Strength | DEcmc |
| 0 | 57.48 | 14.12 | −15.61 | | | |
| 1 | 58.11 | 12.09 | −15.5 | 2.13 | 95.81 | 1.58 |
| 2 | 58.65 | 11.64 | −15.04 | 2.8 | 92.63 | 1.89 |
| 3 | 60.59 | 10.96 | −14.01 | 4.71 | 81.19 | 2.65 |
| 7 | 61.73 | 10.24 | −12.72 | 6.44 | 75.55 | 3.47 |
| 10 | 62.88 | 9.79 | −11.52 | 8.04 | 70.14 | 4.23 |
| 14 | 64.14 | 9.31 | −10.11 | 9.89 | 64.68 | 5.18 |
| 18 | 65.15 | 8.73 | −8.39 | 11.83 | 61.04 | 6.3 |

The L*, a*, b*, DE*, strength and DEcmc values of the powder during refrigerated storage are shown in Table 27.

TABLE 27

| Days in | | | | | Weight | |
|---|---|---|---|---|---|---|
| Storage | L* | a* | b* | DE* | Strength | DEcmc |
| 0 | 57.48 | 14.12 | −15.61 | | | |
| 1 | 56.72 | 13.1 | −16.58 | 1.6 | 104.88 | 1.29 |
| 2 | 56.57 | 12.47 | −16.8 | 2.23 | 105.99 | 1.86 |
| 3 | 57.48 | 12.12 | −16.14 | 2.07 | 99.83 | 1.76 |
| 7 | 57.84 | 11.41 | −16.41 | 2.85 | 97.29 | 2.43 |
| 10 | 57.64 | 11.1 | −16.38 | 3.12 | 98.76 | 2.65 |
| 14 | 58.11 | 11.25 | −15.9 | 2.95 | 95.78 | 2.37 |
| 18 | 58.27 | 10.59 | −15.88 | 3.63 | 94.82 | 2.89 |
| 33 | 58.63 | 11.08 | −15.16 | 3.28 | 92.72 | 2.34 |
| 60 | 59.46 | 10.14 | −14.86 | 4.51 | 87.78 | 3.1 |

### Example 29

A colored powder was made by adding to a WARING® blender 30 g of SIPERNAT® 50 (precipitated silica, commercially available from Evonik Degussa). 5 g of a 2% water emulsion of beta carotene (Product No. 3030; commercially available from Sensient Technologies Corporation) were added drop-wise while blending on a low speed. 10 g of a 50:50 blend of di-alpha tocopherol (commercially available from Roche, Switzerland) and DURKEX® (a partially hydrogenated soybean and cottonseed oil commercially available from Loders Croklaan, Netherlands) were then added drop-wise while blending on low speed. The resulting powder had a yellow color, which was retained after storage in sealed plastic jars at room temperature for 1 week and under refrigeration for 2 weeks.

The L*, a*, b*, DE*, strength and DEcmc values of the powder during storage at room temperature are shown in Table 28.

TABLE 28

| Days in | | | | | Weight | |
|---|---|---|---|---|---|---|
| Storage | L* | a* | b* | DE* | Strength | DEcmc |
| 0 | 85.25 | 12.61 | 56.92 | | | |
| 1 | 84.88 | 12.2 | 56.95 | 0.55 | 102.59 | 0.32 |
| 2 | 84.93 | 11.83 | 57.06 | 0.86 | 102.55 | 0.58 |
| 3 | 85.1 | 11.63 | 57.19 | 1.03 | 101.71 | 0.72 |
| 6 | 85.9 | 10.9 | 56.47 | 1.89 | 93.22 | 1.19 |
| 10 | 86.35 | 9.59 | 54.7 | 3.91 | 83.98 | 2.1 |
| 13 | 86.9 | 8.49 | 53.1 | 5.85 | 75.66 | 2.94 |
| 17 | 87.76 | 7.27 | 51.54 | 7.99 | 66.66 | 3.91 |
| 21 | 87.88 | 5.89 | 49.43 | 10.4 | 60.66 | 5 |
| 36 | 89.13 | 2.11 | 42.07 | 18.6 | 40.85 | 8.45 |

The L*, a*, b*, DE*, strength and DEcmc values of the powder during refrigerated storage are shown in Table 29.

TABLE 29

| Days in | | | | | Weight | |
|---|---|---|---|---|---|---|
| Storage | L* | a* | b* | DE* | Strength | DEcmc |
| 0 | 85.25 | 12.61 | 56.92 | | | |
| 1 | 84.92 | 12.41 | 56.75 | 0.42 | 101.66 | 0.18 |
| 2 | 84.74 | 12.36 | 57.28 | 0.67 | 105 | 0.31 |
| 3 | 84.81 | 12.28 | 57.71 | 0.97 | 106.14 | 0.46 |
| 6 | 85.31 | 11.9 | 57.29 | 0.81 | 100.81 | 0.56 |
| 10 | 85.04 | 11.64 | 57.37 | 1.09 | 102.85 | 0.75 |
| 13 | 85.23 | 11.43 | 57.18 | 1.21 | 100.71 | 0.86 |
| 17 | 85.68 | 11.21 | 57.14 | 1.49 | 97.29 | 1.02 |
| 21 | 85.54 | 10.92 | 56.66 | 1.74 | 96.4 | 1.16 |
| 36 | 85.92 | 10.05 | 55.47 | 3.02 | 89.37 | 1.74 |
| 62 | 86.6 | 8.49 | 53.14 | 5.75 | 77.46 | 2.92 |

### Example 30

A colored powder was made by adding to a WARING® blender 30 g of SIPERNAT® 50 (precipitated silica, commercially available from Evonik Degussa). 10.63 g of a 2% water emulsion of beta carotene (Product No. 3030; commercially available from Sensient Technologies Corporation) were added drop-wise while blending on a low speed. 10.29 g of a 50:50 blend of di-alpha tocopherol (commercially available from Roche, Switzerland) and DURKEX® (a partially hydrogenated soybean and cottonseed oil commercially available from Loders Croklaan, Netherlands) were then added drop-wise while blending on low speed. The resulting powder had a yellow color, which was retained after storage in sealed plastic jars at room temperature 1 week and under refrigeration for at least 5 weeks.

The L*, a*, b*, DE*, strength and DEcmc values of the powder during storage at room temperature are shown in Table 30.

TABLE 30

| Days in | | | | | Weight | |
|---|---|---|---|---|---|---|
| Storage | L* | a* | b* | DE* | Strength | DEcmc |
| 0 | 82.74 | 16.14 | 61.44 | | | |
| 1 | 82.46 | 15.87 | 61.45 | 0.39 | 101.93 | 0.21 |
| 2 | 82.66 | 15.51 | 61.46 | 0.63 | 100.37 | 0.43 |
| 3 | 82.86 | 15.24 | 61.4 | 0.9 | 98.62 | 0.61 |
| 6 | 83.68 | 14.25 | 60.49 | 2.31 | 89.79 | 1.27 |
| 10 | 84.18 | 12.71 | 58.86 | 4.52 | 81.23 | 2.31 |
| 13 | 84.62 | 11.58 | 57.79 | 6.13 | 75.42 | 3.09 |
| 17 | 85.44 | 10.26 | 56.19 | 8.33 | 66.9 | 4.06 |
| 21 | 85.55 | 8.74 | 54.03 | 10.84 | 60.98 | 5.15 |
| 36 | 86.92 | 5.36 | 48.33 | 17.48 | 44.27 | 7.9 |

The L*, a*, b*, DE*, strength and DEcmc values of the powder during refrigerated storage are shown in Table 31.

TABLE 31

| Days in | | | | | Weight | |
|---|---|---|---|---|---|---|
| Storage | L* | a* | b* | DE* | Strength | DEcmc |
| 0 | 82.74 | 16.14 | 61.44 | | | |
| 1 | 82.32 | 16.09 | 61.55 | 0.44 | 103.32 | 0.17 |
| 2 | 82.31 | 16.01 | 61.52 | 0.46 | 103.26 | 0.19 |
| 3 | 82.35 | 16.1 | 61.92 | 0.62 | 104.49 | 0.24 |
| 6 | 82.8 | 15.81 | 61.58 | 0.36 | 99.97 | 0.25 |
| 10 | 82.68 | 15.48 | 61.35 | 0.66 | 99.8 | 0.44 |
| 13 | 82.88 | 15.19 | 61.27 | 0.98 | 98 | 0.64 |
| 17 | 83.36 | 14.96 | 61.22 | 1.35 | 94.59 | 0.82 |
| 21 | 83.06 | 14.76 | 61.1 | 1.45 | 96.02 | 0.92 |
| 36 | 83.75 | 13.54 | 59.52 | 3.38 | 86.01 | 1.73 |
| 62 | 84.58 | 11.99 | 58.2 | 5.58 | 76.91 | 2.81 |

### Example 31

A blend of Polysorbate 80, tocopherol and ascorbyl palmitate was made by mixing equal masses of Polysorbate 80 (48.78% solution; commercially available from BASF, New Jersey, USA) and tocopherol (48.78% solution; commercially available from Roche, Switzerland), and combining 40 g of the blend with 1 g of ascorbyl palmitate.

A colored powder was made by adding to a WARING® blender 30 g of SIPERNAT® 50 (precipitated silica, commercially available from Evonik Degussa). 5 g of a 2% water emulsion of beta carotene (Product No. 3030; commercially available from Sensient Technologies Corporation) were added drop-wise while blending on a low speed. 10 g of the blend of Polysorbate 80, tocopherol and ascorbyl palmitate were then added drop-wise while blending on low speed. The resulting powder had a yellow color, which was retained after storage in sealed plastic jars at room temperature and under refrigeration for at least 5 weeks.

The L*, a*, b*, DE*, strength and DEcmc values of the powder during storage at room temperature are shown in Table 32.

TABLE 32

| Days in | | | | | Weight | |
|---|---|---|---|---|---|---|
| Storage | L* | a* | b* | DE* | Strength | DEcmc |
| 0 | 84.13 | 13.9 | 58.19 | | | |
| 1 | 83.21 | 13.4 | 58.47 | 1.08 | 107.56 | 0.52 |
| 2 | 82.7 | 12.8 | 58.22 | 1.81 | 110.41 | 0.93 |
| 3 | 82.44 | 12.57 | 58 | 2.16 | 111.53 | 1.09 |
| 6 | 82.49 | 12.1 | 57.79 | 2.48 | 110.26 | 1.36 |
| 10 | 82.26 | 11.49 | 57.32 | 3.18 | 109.94 | 1.75 |
| 13 | 82.29 | 10.99 | 56.89 | 3.69 | 107.74 | 2.06 |
| 17 | 82.87 | 10.43 | 56.31 | 4.14 | 101.13 | 2.37 |
| 21 | 82.79 | 9.76 | 55.25 | 5.25 | 97.57 | 2.83 |
| 36 | 83.56 | 8.06 | 52.23 | 8.36 | 82.06 | 4.11 |
| 63 | 86.39 | 5.52 | 45.05 | 15.75 | 50.69 | 6.71 |

The L*, a*, b*, DE*, strength and DEcmc values of the powder during refrigerated storage are shown in Table 33.

TABLE 33

| Days in | | | | | Weight | |
|---|---|---|---|---|---|---|
| Storage | L* | a* | b* | DE* | Strength | DEcmc |
| 0 | 84.13 | 13.9 | 58.19 | | | |
| 1 | 83.65 | 13.85 | 58.56 | 0.61 | 104.78 | 0.23 |
| 2 | 83.63 | 13.85 | 58.93 | 0.9 | 106.39 | 0.35 |
| 3 | 83.48 | 13.84 | 59.18 | 1.19 | 108.55 | 0.46 |
| 6 | 83.7 | 13.59 | 59.03 | 1 | 106.27 | 0.47 |
| 10 | 83.05 | 13.19 | 58.81 | 1.44 | 110.23 | 0.73 |
| 13 | 82.95 | 13.07 | 58.84 | 1.58 | 111.1 | 0.82 |
| 17 | 83.18 | 12.95 | 58.96 | 1.55 | 109.9 | 0.87 |
| 21 | 82.76 | 12.71 | 58.74 | 1.89 | 112.08 | 1.04 |
| 36 | 82.67 | 12.14 | 58.23 | 2.29 | 110.73 | 1.34 |
| 63 | 82.23 | 14.5 | 59.53 | 2.41 | 118.94 | 0.88 |

### Example 32

A colored powder was made by adding to a WARING® blender 30 g of SIPERNAT® 50 (precipitated silica, commercially available from Evonik Degussa). 10 g of a 2% water emulsion of beta carotene (Product No. 3030; commercially available from Sensient Technologies Corporation) were added drop-wise while blending on a low speed. 10.5 g of the blend of Polysorbate 80, tocopherol and ascorbyl palmitate from Example 31 were then added drop-wise while blending on low speed. The resulting powder had a yellow color, which was retained after storage in sealed plastic jars at room temperature for 2 weeks and under refrigeration for at least 5 weeks.

The L*, a*, b*, DE*, strength and DEcmc values of the powder during storage at room temperature are shown in Table 34.

TABLE 34

| Days in | | | | | Weight | |
|---|---|---|---|---|---|---|
| Storage | L* | a* | b* | DE* | Strength | DEcmc |
| 0 | 81.7 | 17.56 | 62.13 | | | |
| 1 | 80.92 | 17.29 | 61.97 | 0.84 | 104.8 | 0.33 |
| 2 | 80.57 | 16.68 | 61.61 | 1.52 | 105.95 | 0.7 |
| 3 | 80.39 | 16.6 | 61.43 | 1.77 | 106.53 | 0.78 |
| 6 | 80.9 | 16.02 | 61.15 | 1.99 | 101.6 | 1.03 |
| 10 | 81.37 | 14.48 | 59.65 | 3.96 | 92.54 | 2 |
| 13 | 81.81 | 13.43 | 58.55 | 5.46 | 85.93 | 2.7 |
| 17 | 82.67 | 12.43 | 57.45 | 7.01 | 77.47 | 3.41 |
| 21 | 82.89 | 11.31 | 55.92 | 8.89 | 71.95 | 4.21 |
| 36 | 84.4 | 8.49 | 51.09 | 14.54 | 53.81 | 6.43 |
| 63 | 85.27 | 5.74 | 47.5 | 19.14 | 44.03 | 8.54 |

The L*, a*, b*, DE*, strength and DEcmc values of the powder during refrigerated storage are shown in Table 35.

TABLE 35

| Days in | | | | | Weight | |
|---|---|---|---|---|---|---|
| Storage | L* | a* | b* | DE* | Strength | DEcmc |
| 0 | 81.7 | 17.56 | 62.13 | | | |
| 1 | 81.27 | 17.53 | 62.2 | 0.43 | 103.25 | 0.16 |
| 2 | 81.29 | 17.64 | 62.61 | 0.64 | 104.74 | 0.23 |
| 3 | 81.18 | 17.68 | 62.45 | 0.62 | 104.9 | 0.22 |
| 6 | 81.37 | 17.53 | 62.59 | 0.56 | 104.05 | 0.22 |
| 10 | 81.06 | 17.25 | 62.19 | 0.72 | 104.74 | 0.32 |
| 13 | 81.04 | 17.3 | 62.52 | 0.81 | 106.2 | 0.36 |
| 17 | 81.46 | 17.13 | 62.26 | 0.51 | 102.18 | 0.33 |
| 21 | 81.12 | 17.02 | 62.12 | 0.8 | 103.97 | 0.43 |
| 36 | 81.42 | 16.26 | 61.52 | 1.46 | 99.3 | 0.85 |
| 63 | 82.76 | 11.4 | 57.4 | 7.83 | 77.63 | 4.07 |

### Example 33

A colored powder was made by adding to a WARING® blender 30 g of SIPERNAT® 50 (precipitated silica, commercially available from Evonik Degussa). 15.5 g of a 2% water emulsion of beta carotene (Product No. 3030; commercially available from Sensient Technologies Corporation) were added drop-wise while blending on a low speed. 20 g of a blend of 70% di alpha tocopherol (commercially available from Roche) and 30% soybean oil (commercially available from Central Soya) were then added drop-wise while blending on low speed. The resulting powder had a yellow color, which was retained after storage in sealed plastic jars at room temperature for 1 week and under refrigeration for 2 weeks.

The L*, a*, b*, DE*, strength and DEcmc values of the powder during storage at room temperature are shown in Table 36.

TABLE 36

| Days in | | | | | Weight | |
|---|---|---|---|---|---|---|
| Storage | L* | a* | b* | DE* | Strength | DEcmc |
| 0 | 82.14 | 15.98 | 57.96 | | | |
| 1 | 82.51 | 15.57 | 58.3 | 0.65 | 98.38 | 0.38 |
| 4 | 83.82 | 13.25 | 56.88 | 3.39 | 84.43 | 1.94 |
| 8 | 84.69 | 10.04 | 53.3 | 7.97 | 69.03 | 4.14 |
| 11 | 85.49 | 7.95 | 50.66 | 11.36 | 58.84 | 5.7 |
| 15 | 86.54 | 6.35 | 48.51 | 14.2 | 50.13 | 6.98 |

The L*, a*, b*, DE*, strength and DEcmc values of the powder during refrigerated storage are shown in Table 37.

TABLE 37

| Days in | | | | | Weight | |
|---|---|---|---|---|---|---|
| Storage | L* | a* | b* | DE* | Strength | DEcmc |
| 0 | 82.14 | 15.98 | 57.96 | | | |
| 1 | 82.24 | 16.03 | 58.44 | 0.49 | 101.02 | 0.18 |
| 4 | 82.65 | 15.93 | 59.05 | 1.2 | 100.21 | 0.48 |
| 8 | 83.05 | 14.61 | 57.78 | 1.65 | 92.63 | 1 |
| 11 | 83.53 | 13.78 | 57.12 | 2.74 | 87.21 | 1.57 |
| 15 | 84.02 | 13.19 | 56.96 | 3.52 | 83.55 | 2.01 |
| 19 | 83.87 | 12.6 | 56.45 | 4.09 | 82.66 | 2.36 |
| 34 | 84.75 | 9.96 | 54.08 | 7.62 | 70.78 | 4.18 |
| 61 | 86.22 | 6.34 | 49.74 | 13.32 | 53.83 | 6.87 |

### Example 34

A colored powder was made by adding to a WARING® blender 30 g of SIPERNAT® 50 (precipitated silica, commercially available from Evonik Degussa). 20 g of a 2% water emulsion of beta carotene (Product No. 3030; commercially available from Sensient Technologies Corporation) were added drop-wise while blending on a low speed. 10.72 g of a blend of 25% Ascorbic Acid solution were then added drop-wise while blending on low speed. 7.44 μm of a 20% Quick Coat solution (commercially available from Alfred L Wolff) were then added drop-wise while blending on low speed. The resulting powder had a yellow color, which was retained after storage at room temperature for 1 week and under refrigeration for 1 week.

The L*, a*, b*, DE*, strength and DEcmc values of the powder during storage at room temperature are shown in Table 38.

TABLE 38

| Days in | | | | | Weight | |
|---|---|---|---|---|---|---|
| Storage | L* | a* | b* | DE* | Strength | DEcmc |
| 0 | 79.67 | 20.21 | 55.74 | | | |
| 2 | 79.13 | 18.92 | 48.85 | 7.03 | 80.33 | 2.67 |
| 5 | 78.71 | 19.2 | 48.44 | 7.44 | 81.5 | 2.92 |
| 7 | 79.15 | 18.89 | 48.28 | 7.6 | 78.55 | 2.91 |
| 9 | 79.79 | 18.42 | 47.86 | 8.08 | 73.94 | 3.01 |
| 12 | 79.39 | 17.94 | 46.91 | 9.13 | 73.43 | 3.36 |
| 16 | 79.21 | 16.67 | 44.75 | 11.56 | 68.79 | 4.19 |
| 19 | 79.37 | 16.48 | 43.65 | 12.66 | 65.37 | 4.6 |
| 23 | 78.91 | 16.29 | 42.61 | 13.72 | 65.09 | 5 |
| 27 | 79.24 | 15.49 | 40.14 | 16.31 | 58.14 | 5.94 |
| 41 | 79.44 | 15.27 | 37.79 | 18.62 | 52.68 | 6.84 |

The L*, a*, b*, DE*, strength and DEcmc values of the powder during refrigerated storage are shown in Table 39.

TABLE 39

| Days in | | | | | Weight | |
|---|---|---|---|---|---|---|
| Storage | L* | a* | b* | DE* | Strength | DEcmc |
| 0 | 79.67 | 20.21 | 55.74 | | | |
| 2 | 78.52 | 19.98 | 49.45 | 6.4 | 85.76 | 2.71 |
| 5 | 78.88 | 19.19 | 48.86 | 7 | 81.82 | 2.73 |
| 7 | 78.76 | 19.11 | 48.15 | 7.73 | 80.38 | 3.02 |
| 9 | 78.57 | 19.08 | 48.42 | 7.49 | 82.24 | 2.9 |
| 12 | 79.24 | 19.09 | 47.93 | 7.91 | 77.13 | 3.09 |
| 16 | 78.63 | 19.23 | 48.02 | 7.85 | 80.77 | 3.11 |
| 19 | 78.99 | 18.94 | 47.49 | 8.38 | 77.24 | 3.26 |
| 23 | 78.98 | 19.16 | 47.77 | 8.07 | 78.08 | 3.19 |
| 27 | 78.59 | 19.11 | 47.53 | 8.36 | 79.48 | 3.29 |
| 41 | 79.38 | 18.21 | 47.08 | 8.9 | 74.02 | 3.31 |
| 69 | 79.29 | 18.06 | 46.49 | 9.51 | 72.88 | 3.54 |

### Example 35

A colored powder is made by adding to a WARING® blender 15 g of SIPERNAT® 50 (precipitated silica, commercially available from Evonik Degussa) and 15 g of N-Zorbit. 10 g of a 2% water emulsion of beta carotene (Product No. 3030; commercially available from Sensient Technologies Corporation) are added drop-wise while blending on a low speed. 20 g of a blend of 70% di alpha tocopherol (commercially available from Roche) and 30% soybean oil (commercially available from Central Soya) are then added drop-wise while blending on low speed. The resulting powder is expected to have a yellow color which is expected to be retained after refrigerated storage for a period of 6 months.

### Example 36

A colored powder is made by adding to a WARING® blender 30 g of SIPERNAT® 50 (precipitated silica, commercially available from Evonik Degussa). 5 g of a 10% tetrasodium pyrophosphate solution (commercially available from ICL Performance Products) and 5 g of a 5% solution of potassium carbonate (commercially available from Univar) are then added drop-wise while blending on low speed. 15 g of a red cabbage solution (Product No. 3810; commercially available from Sensient Technologies Corporation) are added drop-wise while blending on a low speed. The resulting powder is expected to have a purple to blue color, which is expected to be retained after storage at room temperature and under refrigerated storage for 2 weeks to 6 months.

### Example 37

A colored powder is made by adding to a WARING® blender 30 g of SIPERNAT® 50 (precipitated silica, commercially available from Evonik Degussa). 5 g of a 10% tetrasodium pyrophosphate solution (commercially available from ICL Performance Products) and 5 g of a 5% solution of potassium carbonate (commercially available from Univar) are then added drop-wise while blending on low speed. 15 g of a purple sweet potato solution with E=30% (a more concentrated form of Product No. 3805; commercially available from Sensient Technologies Corporation) are added drop-wise while blending on a low speed. The resulting powder is expected to have a purple to blue color, which is expected to be retained after storage at room temperature and under refrigerated storage for 2 weeks to 6 months.

### Example 38

A colored powder is made by adding to a WARING® blender 30 g of SIPERNAT® 50 (precipitated silica, commercially available from Evonik Degussa). 5 g of a 10% tetrasodium pyrophosphate solution (commercially available from ICL Performance Products) and 5 g of a 5% solution of potassium carbonate (commercially available from Univar) are then added drop-wise while blending on low speed. 15 g of a E1=8-10% black carrot anthocyanin solution (Product No. 3847; commercially available from Sensient Technologies Corporation) are added drop-wise while blending on a low speed. The resulting powder is expected to have a purple color, which is expected to be retained after storage at room temperature and under refrigerated storage for 2 weeks to 6 months.

### Example 39

A colored powder is made by adding to a WARING® blender 30 g of SIPERNAT® 50 (precipitated silica, commercially available from Evonik Degussa). 5 g of a 5% solution of potassium carbonate (commercially available from Univar) are then added drop-wise while blending on low speed. 15 g of a red cabbage solution (Product No. 3810; commercially available from Sensient Technologies Corporation) are added drop-wise while blending on a low speed. The resulting powder is expected to have a purple to blue color, which is expected to be retained after storage at room temperature and refrigerated for 2 weeks to 6 months.

### Example 40

A colored powder is made by adding to a WARING® blender 30 g of SIPERNAT® 50 (precipitated silica, commercially available from Evonik Degussa). 5 g of a 5% solution of potassium carbonate (commercially available from Univar) are then added drop-wise while blending on low speed. 15 g of a purple sweet potato solution with E=30% (a more concentrated form of Product No. 3805; commercially available from Sensient Technologies Corporation) are added drop-wise while blending on a low speed. The resulting powder is expected to have a purple to blue color, which is expected to be retained after storage at room temperature and refrigerated for 2 weeks to 6 months.

### Example 41

A colored powder is made by adding to a WARING® blender 30 g of SIPERNAT® 50 (precipitated silica, commercially available from Evonik Degussa). 5 g of a 5% solution of potassium carbonate (commercially available from Univar) are then added drop-wise while blending on low speed. 15 g of a E1=8-10% black carrot anthocyanin solution (Product No. 3847; commercially available from Sensient Technologies Corporation) are added drop-wise while blending on a low speed. The resulting powder is expected to have a purple color, which is expected to be retained after storage at room temperature and refrigerated for 2 weeks to 6 months.

### Example 42

A colored powder is made by adding to a WARING® blender 15 g of SIPERNAT® 50 (precipitated silica, commercially available from Evonik Degussa) and 15 g of N-Zorbit. 10 g of a 2% water emulsion of beta carotene (Product No. 3030; commercially available from Sensient Technologies Corporation) are added drop-wise while blending on a low speed. 20 g of a blend of 70% di alpha tocopherol (commercially available from Roche) and 30% soybean oil (commercially available from Central Soya) are then added drop-wise while blending on low speed. The colored powder is divided into two batches and coated with either a cellulose or a fumed silica. The resulting powder is expected to have a yellow color which is expected to be retained after refrigerated storage for a period of 6 months.

### Example 43

A colored powder is made by adding to a WARING® blender 30 g of SIPERNAT® 50 (precipitated silica, commercially available from Evonik Degussa). 5 g of a 10% tetrasodium pyrophosphate solution (commercially available from ICL Performance Products) and 5 g of a 5% solution of potassium carbonate (commercially available from Univar) are then added drop-wise while blending on low speed. 15 g of a red cabbage solution (Product No. 3810; commercially available from Sensient Technologies Corporation) are added drop-wise while blending on a low speed. The colored powder is divided into two batches and coated with either a cellulose or fumed silica. The resulting powder is expected to have a purple to blue color, which is expected to be retained after storage at room temperature or under refrigeration for a period of 2 weeks to 6 months.

### Example 44

A colored powder is made by adding to a WARING® blender 30 g of SIPERNAT® 50 (precipitated silica, commercially available from Evonik Degussa). 5 g of a 10% tetrasodium pyrophosphate solution (commercially available from ICL Performance Products) and 5 g of a 5% solution of potassium carbonate (commercially available from Univar) are then added drop-wise while blending on low speed. 15 g of a purple sweet potato solution with E=30% (a more concentrated form of Product No. 3805; commercially available from Sensient Technologies Corporation) are added drop-wise while blending on a low speed. The colored powder is divided into two batches and coated with either a cellulose or fumed silica. The resulting powder is expected to have a purple to blue color, which is expected to be retained after storage at room temperature or under refrigeration for a period of 2 weeks to 6 months.

### Example 45

A colored powder is made by adding to a WARING® blender 30 g of SIPERNAT® 50 (precipitated silica, commercially available from Evonik Degussa). 5 g of a 10% tetrasodium pyrophosphate solution (commercially available from ICL Performance Products) and 5 g of a 5% solution of potassium carbonate (commercially available from Univar) are then added drop-wise while blending on low speed. 15 g of a E1=8-10% black carrot anthocyanin solution (Product No. 3847; commercially available from Sensient Technologies Corporation) were added drop-wise while blending on a low speed. The colored powder is divided into two batches and coated with either a cellulose or fumed silica. The resulting powder is expected to have a purple color, which is expected to be retained after storage at room temperature or under refrigeration for a period of 2 weeks to 6 months.

### Example 46

A colored powder is made by adding to a WARING® blender 30 g of SIPERNAT® 50 (precipitated silica, commercially available from Evonik Degussa). 5 g of a 5% solution of potassium carbonate (commercially available from Univar) are then added drop-wise while blending on low speed. 15 g of a red cabbage solution (Product No. 3810; commercially available from Sensient Technologies Corporation) are added drop-wise while blending on a low speed. The colored powder is divided into two batches and coated with either a cellulose or fumed silica. The resulting powder is expected to have a purple to blue color, which is expected to be retained after storage at room temperature or under refrigeration for 2 weeks to 6 months.

### Example 47

A colored powder is made by adding to a WARING® blender 30 g of SIPERNAT® 50 (precipitated silica, commercially available from Evonik Degussa). 5 g of a 5% solution of potassium carbonate (commercially available from Univar) are then added drop-wise while blending on low speed. 15 g of a purple sweet potato solution with E=30% (a more concentrated form of Product No. 3805; commercially available from Sensient Technologies Corporation) are added drop-wise while blending on a low speed. The colored powder is divided into two batches and coated with either a cellulose or fumed silica. The resulting powder is expected to have a purple to blue color, which is expected to be retained after storage at room temperature or under refrigeration for 2 weeks to 6 months.

### Example 48

A colored powder is made by adding to a WARING® blender 30 g of SIPERNAT® 50 (precipitated silica, commercially available from Evonik Degussa). 5 g of a 5% solution of potassium carbonate (commercially available from Univar) are then added drop-wise while blending on low speed. 15 g of a E1=8-10% black carrot anthocyanin solution (Product No. 3847; commercially available from Sensient Technologies Corporation) are added drop-wise while blending on a low speed. The colored powder is divided into two batches and coated with either a cellulose or fumed silica. The resulting powder is expected to have a purple color, which is expected to be retained after storage at room temperature or under refrigeration for 2 weeks to 6 months.

### Example 49

69.8 g of dextrose is mixed with 25 g sucrose and 5 g of malic acid. 0.2 g of the colored powder of Example 13 is added and mixed in by stirring. Compressed confectionary tablets are formed by placing portions of the mixture into a dye, the dye is leveled to the top of the dye and pressure is applied at 2000-4000 lbs of force. The confectionary tablets are expected to have a pink color that is retained upon storage for several months at room temperature and/or refrigeration.

### Example 50

94.2 g of dextrose and 5 g citric acid are mixed together. 0.8 g of the colored powder from Example 31 is added and mixed in by shaking. Compressed confectionary tablets are formed by placing portions of the mixture into a dye. The dye is leveled to the top of the dye and pressure is applied at 2000-4000 lbs of force. The confectionary tablets are expected to have a light yellow color that is retained upon storage for several months at room temperature and/or refrigeration.

### Example 51

0.2 g of the colored powder from Example 13 and 0.8 g of the colored powder from Example 31 are blended with 99 g of a spice mix having a maltodextrin base and 5% salt with additional spices and natural flavorings to form a colored seasoning mix. The seasoning mix is sprinkled onto potato chips and crackers to produce flavored and colored potato chips and crackers. The seasoning mix and the coated potato chips and crackers are expected to have a good orange color that is retained upon storage for several months at room temperature.

### Example 52

The colored powder is incorporated in a compressed nutraceutical tablet by blending 99.8 g of calcium carbonate and 0.2 g of the colored powder from Example 25. The powder is placed into a dye, the dye is leveled to the top of the dye and pressure is applied at 2000-4000 lbs of force to form compressed tablets. The tablets are expected to have a purple color that is retained upon storage for several months at room temperature and/or refrigeration.

### Example 52

The colored powder of Example 13 is mixed with a lactose filler, a cellulose binder and a pharmaceutical such that the colored powder is incorporated at 0.15% by weight of the total mixture. The mixture is wet granulated and dried. A compressed colored pharmaceutical tablet is formed by placing portions of the mixture into a dye. The dye is leveled to the top of the dye and pressure is applied at 2000-4000 lbs of force. The compressed pharmaceutical tablets are expected to have a pink color that is retained upon storage for several months at room temperature and/or refrigeration.

### Example 53

A colored powder was made by adding to a Littleford mixer 4.95 lb of SIPERNAT® 50 (precipitated silica, commercially available from Evonik Degussa). 0.92 lb of a 50% solution of citric acid were then added drop-wise to the mixture blending on a low speed. 0.5 lb of di-alpha tocopherol (commercially available from Roche, Switzerland) were then added drop-wise while blending on low speed. 3.63 lb of a purple sweet potato solution (a more concentrated form of Product No. 3805; commercially available from Sensient Technologies Corporation) were then added drop-wise while mixing on a low speed. The mixture was dried while blending for 90 minutes by supplying steam at 15 psi to the jacket of the mixer at a temperature of 50° C. The resulting powder had a moisture content of 9.2% and a good even red color, which was retained after storage at room temperature and under refrigeration in sealed plastic jars for at least one month.

TABLE 40

| Days in | | | | | | |
|---|---|---|---|---|---|---|
| Storage | Delta Description | L* | a* | b* | DE* | DEcmc |
| 0 | Darker, less red, less | 33.87 | 48.76 | 1.2 | | |
| 14 | blue | 33.7 | 48.58 | 1.36 | 0.3 | 0.15 |

### Example 54

A colored powder was made by adding to a Littleford mixer 6.023 lb of SIPERNAT® 50 (precipitated silica, commercially available from Evonik Degussa). 9.19 lb of a 6% tetrasodium pyrophosphate solution and 0.2 lb of 50% solution of potassium carbonate were added drop-wise while mixing on low speed. 2.446 lb of a purple sweet potato solution (a more concentrated form of Product No. 3805; commercially available from Sensient Technologies Corporation) and 0.412 lb of di-alpha tocopherol (commercially available from Roche, Switzerland) were then added drop-wise while mixing on a low speed. The mixture was dried while blending for 90 minutes by supplying steam at 15 psi to the jacket of the mixer at a temperature of 50° C. The resulting powder had a moisture content of 3.8% and a purple color, which was retained after storage in sealed plastic jars at room temperature and under refrigeration for at least one month

TABLE 41

| Days in | | | | | | |
|---|---|---|---|---|---|---|
| Storage | Delta Description | L* | a* | b* | DE* | DEcmc |
| 0 | Darker, redder, less | 34.01 | 21.48 | −21.21 | | |
| 14 | blue | 33.46 | 21.63 | −21.05 | 0.59 | 0.35 |

### Example 55

A colored powder was made by adding to a Littleford mixer 5.346 lb of SIPERNAT® 50 (precipitated silica, commercially available from Evonik Degussa). 1.858 lb of a 2% water emulsion of beta carotene (Product No. 3041; commercially available from Sensient Technologies Corporation) and 1.789 lb of turmeric liquid (Product No. 3285; commercially available from Sensient Technologies Corporation) were added drop-wise while blending on a low speed. 0.75 lb of Polysorbate 80 and 0.75 lb tocopherol were then added drop-wise while mixing on low speed. The resulting powder had a moisture content of 5.5% and a yellow color, which was retained after storage at room temperature and under refrigeration in sealed plastic jars for at least one month.

TABLE 42

| Days in | | | | | | |
|---|---|---|---|---|---|---|
| Storage | Delta Description | L* | a* | b* | DE* | DEcmc |
| 0 | Darker, redder, more | 71.43 | 20.84 | 85.95 | | |
| 14 | yellow | 71.21 | 21.07 | 87.35 | 1.44 | 0.45 |

All patents, publications and references cited herein are hereby fully incorporated by reference. In case of conflict between the present disclosure and incorporated patents, publications and references, the present disclosure should control.

What is claimed is:

1. A colored powder comprising silica and a soluble dye, wherein the powder is substantially free of aluminum salts.

2. The powder of claim 1, wherein the soluble dye comprises a natural dye.

3. The powder of claim 2, wherein the natural dye comprises a dye selected from an anthocyanin, beta carotene, turmeric, betanin and combinations thereof.

4. The powder of claim 3, wherein the natural dye comprises an anthocyanin.

5. The powder of claim 1, wherein the dye is adsorbed onto the silica.

6. The powder of claim 1, wherein the silica has an average particle size of at least about 4 μm.

7. The powder of claim 1, further comprising a component selected from citric acid, tocopherol, ascorbic acid, sodium hexametaphosphate and combinations thereof.

8. The powder of claim 1, wherein the colored powder has a color and the color is retained after storage at 4° C. for at least about one month.

9. The powder of claim 1, wherein the powder comprises from about 0.1% (dsb) to about 5% (dsb) food grade dye.

10. A method of coloring an edible substrate comprising plating the colored powder of claim 1 onto the substrate.

11. An edible solid substrate comprising an edible substrate and colored powder comprising silica and a soluble dye adsorbed onto the silica.

12. The edible solid substrate of claim 11, wherein the colored powder is plated onto the edible substrate.

13. The edible solid substrate of claim 11, wherein the edible substrate comprises one or more of a tablet, candy, fabricated snack, capsule, pet food, dessert, snack chip, baked product, fruit, cheese, processed meat, pasta and oral hygiene product.

14. The edible solid substrate of claim 11, wherein the edible substrate comprises a pharmaceutical.

15. The edible solid substrate of claim 11, wherein the edible substrate comprises a nutraceutical.

16. The edible solid substrate of claim 11, wherein the edible substrate comprises a plurality of edible particles.

17. The edible solid substrate of claim 11, wherein the edible substrate comprises a compressed tablet.

18. The edible solid substrate of claim 11, wherein the soluble dye comprises a natural dye.

19. The edible solid substrate of claim 11, wherein the colored powder is substantially free of aluminum salts.

20. A method of coloring an edible substrate, the method comprising combining a colored powder comprising silica and a soluble dye adsorbed onto the silica with an edible substrate.

21. The method of claim 20, wherein the edible substrate comprises edible particles.

22. The method of claim 20, wherein the L* value of the dye adsorbed onto the silica is at least about 10% greater than the L* value of the dye before being adsorbed onto the silica.

23. The method of claim 20, wherein the soluble dye comprises a natural dye.

24. The method of claim 20, wherein the colored powder is substantially free of aluminum salts.

25. The method of claim 20, wherein the silica comprises precipitated silica, gel silica, fumed silica, or a combination thereof.

26. A method of increasing the color intensity of a dye, the method comprising
adsorbing a solution of a soluble dye onto silica powder to form a colored powder; and
drying the colored powder using heat, a vacuum or a combination thereof, thereby increasing the color intensity of the dye.

27. The method of claim 26, wherein the soluble dye comprises a natural dye.

28. The method of claim 26, wherein the L* value of the dye adsorbed onto the silica is at least about 10% greater than the L* value of the dye before being adsorbed onto the silica.

29. The method of claim 26, wherein the color of the colored powder is retained after storage at 4° C. for at least about one month.

30. The method of claim 26, wherein the colored powder is stable after storage at 4° C. for at least about one month.
